SEQUENCE LISTING (94 sequences)

SEQ ID NO: 1 - protein, 283 aa, Homo sapiens
MAVPPTYADLGKSARDVFTKGYGFGLIKLDLKTKSENGLEFTSSGSANTETTKVTGSLETKYRWTEYGLTFTEKWNTDNTLGTEITVEDQLARGLKLTFDSSFSPNTGKKNAKIKTGYKREHINLGCDMDFDIAGPSIRGALVLGYEGWLAGYQMNFETAKSRVTQSNFAVGYKTDEFQLHTNVNDGTEFGGSIYQKVNKKLETAVNLAWTAGNSNTRFGIAAKYQIDPDACFSAKVNNSSLIGLGYTQTLKPGIKLTLSALLDGKNVNAGGHKLGLGLEFQA

SEQ ID NO: 2 - DNA, 852 bp, Homo sapiens
  source 1..852: mol_type = genomic DNA
atggctgtgccacccacgtatgccgatcttggcaaatctgccagggatgtcttcaccaagggctatggatttggcttaataaagcttgatttgaaaacaaaatctgagaatggattggaatttacaagctcaggctcagccaacactgagaccaccaaagtgacgggcagtctggaaaccaagtacagatggactgagtacggcctgacgtttacagagaaatggaataccgacaatacactaggcaccgagattactgtggaagatcagcttgcacgtggactgaagctgaccttcgattcatccttctcacctaacactgggaaaaaaaatgctaaaatcaagacagggtacaagcgggagcacattaacctgggctgcgacatggatttcgacattgctgggccttccatccggggtgctctggtgctaggttacgagggctggctggccggctaccagatgaattttgagactgcaaaatcccgagtgacccagagcaactttgcagttggctacaagactgatgaattccagcttcacactaatgtgaatgacgggacagagtttggcggctccatttaccagaaagtgaacaagaagttggagaccgctgtcaatcttgcctggacagcaggaaacagtaacacgcgcttcggaatagcagccaagtatcagattgaccctgacgcctgcttctcggctaaagtgaacaactccagcctgataggtttaggatacactcagactctaaagccaggtattaaactgacactgtcagctcttctggatggcaagaacgtcaatgctggtggccacaagcttggtctaggactggaatttcaagcataa

SEQ ID NO: 3 - RNA, 19 nt, synthetic construct
  misc_feature 1..19: note = Synthetic oligonucleotide
  source 1..19: mol_type = other RNA
acactaggcaccgagatta

SEQ ID NO: 4 - RNA, 19 nt, synthetic construct
  misc_feature 1..19: note = Synthetic oligonucleotide
  source 1..19: mol_type = other RNA
gggctatggatttggctta

SEQ ID NO: 5 - RNA, 19 nt, synthetic construct
  misc_feature 1..19: note = Synthetic oligonucleotide
  source 1..19: mol_type = other RNA
gcttggtctaggactggaa

SEQ ID NO: 6 - RNA, 19 nt, synthetic construct
  misc_feature 1..19: note = Synthetic oligonucleotide
  source 1..19: mol_type = other RNA
aagctgaccttcgattcat

SEQ ID NO: 7 - RNA, 19 nt, synthetic construct
  misc_feature 1..19: note = Synthetic oligonucleotide
  source 1..19: mol_type = other RNA
gaatgacgggacagagttt

SEQ ID NO: 8 - RNA, 19 nt, synthetic construct
  misc_feature 1..19: note = Synthetic oligonucleotide
  source 1..19: mol_type = other RNA
tcggaatagcagccaagta

SEQ ID NO: 9 - RNA, 19 nt, synthetic construct
  misc_feature 1..19: note = Synthetic oligonucleotide
  source 1..19: mol_type = other RNA
ctcttctggatggcaagaa

SEQ ID NO: 10 - RNA, 20 nt, synthetic construct
  misc_feature 1..20: note = Synthetic oligonucleotide
  source 1..20: mol_type = other RNA
gaatagcagccaagtatcag

SEQ ID NO: 11 - RNA, 19 nt, synthetic construct
  misc_feature 1..19: note = Synthetic oligonucleotide
  source 1..19: mol_type = other RNA
taagccaaatccatagccc

SEQ ID NO: 12 - RNA, 19 nt, synthetic construct
  misc_feature 1..19: note = Synthetic oligonucleotide
  source 1..19: mol_type = other RNA
ttccagtcctagaccaagc

SEQ ID NO: 13 - RNA, 19 nt, synthetic construct
  misc_feature 1..19: note = Synthetic oligonucleotide
  source 1..19: mol_type = other RNA
tgatacttggctgctattc

SEQ ID NO: 14 - RNA, 19 nt, synthetic construct
  misc_feature 1..19: note = Synthetic oligonucleotide
  misc_feature 5: note = 2'-O-methyl modification
  misc_feature 8: note = 2'-O-methyl modification
  misc_feature 13: note = 2'-O-methyl modification
  misc_feature 17: note = 2'-O-methyl modification
  source 1..19: mol_type = other RNA
acactaggcaccgagatta

SEQ ID NO: 15 - RNA, 19 nt, synthetic construct
  misc_feature 1..19: note = Synthetic oligonucleotide
  misc_feature 6: note = 2'-O-methyl modification
  misc_feature 16: note = 2'-O-methyl modification
  source 1..19: mol_type = other RNA
taatctcggtgcctagtgt

SEQ ID NO: 16 - DNA, 19 bp, synthetic construct
  misc_feature 1..19: note = Synthetic oligonucleotide
  source 1..19: mol_type = other DNA
acactaggcaccgagatta

SEQ ID NO: 17 - DNA, 19 bp, synthetic construct
  misc_feature 1..19: note = Synthetic oligonucleotide
  source 1..19: mol_type = other DNA
gggctatggatttggctta

SEQ ID NO: 18 - DNA, 19 bp, synthetic construct
  misc_feature 1..19: note = Synthetic oligonucleotide
  source 1..19: mol_type = other DNA
gcttggtctaggactggaa

SEQ ID NO: 19 - DNA, 19 bp, synthetic construct
  misc_feature 1..19: note = Synthetic oligonucleotide
  source 1..19: mol_type = other DNA
aagctgaccttcgattcat

SEQ ID NO: 20 - DNA, 19 bp, synthetic construct
  misc_feature 1..19: note = Synthetic oligonucleotide
  source 1..19: mol_type = other DNA
gaatgacgggacagagttt

SEQ ID NO: 21 - DNA, 19 bp, synthetic construct
  misc_feature 1..19: note = Synthetic oligonucleotide
  source 1..19: mol_type = other DNA
tcggaatagcagccaagta

SEQ ID NO: 22 - DNA, 19 bp, synthetic construct
  misc_feature 1..19: note = Synthetic oligonucleotide
  source 1..19: mol_type = other DNA
ctcttctggatggcaagaa

SEQ ID NO: 23 - DNA, 20 bp, synthetic construct
  misc_feature 1..20: note = Synthetic oligonucleotide
  source 1..20: mol_type = other DNA
gaatagcagccaagtatcag

SEQ ID NO: 24 - RNA, 19 nt, synthetic construct
  misc_feature 1..19: note = Synthetic oligonucleotide
  misc_feature 8: note = 2'-O-methyl-modification
  misc_feature 13: note = 2'-O-methyl-modification
  misc_feature 17: note = 2'-O-methyl-modification
  source 1..19: mol_type = other RNA
gcaaacatcccagaggtat

SEQ ID NO: 25 - RNA, 19 nt, synthetic construct
  misc_feature 1..19: note = Synthetic oligonucleotide
  misc_feature 6: note = 2'-O-methyl-modification
  misc_feature 11: note = 2'-O-methyl-modification
  source 1..19: mol_type = other RNA
atacctctgggatgtttgc

SEQ ID NO: 26 - DNA, 20 bp, synthetic construct
  misc_feature 1..20: note = Primer
  source 1..20: mol_type = other DNA
actcttccagccttccttcc

SEQ ID NO: 27 - DNA, 20 bp, synthetic construct
  misc_feature 1..20: note = Primer
  source 1..20: mol_type = other DNA
tgttggcgtacaggtctttg

SEQ ID NO: 28 - DNA, 20 bp, synthetic construct
  misc_feature 1..20: note = Primer
  source 1..20: mol_type = other DNA
ccaaagtcctggaggttgaa

SEQ ID NO: 29 - DNA, 20 bp, synthetic construct
  misc_feature 1..20: note = Primer
  source 1..20: mol_type = other DNA
taactccaggccatcacaca

SEQ ID NO: 30 - DNA, 21 bp, synthetic construct
  misc_feature 1..21: note = Primer
  source 1..21: mol_type = other DNA
agtttccagctgggagaagag

SEQ ID NO: 31 - DNA, 21 bp, synthetic construct
  misc_feature 1..21: note = Primer
  source 1..21: mol_type = other DNA
ctttgccatcccatttctgta

SEQ ID NO: 32 - DNA, 20 bp, synthetic construct
  misc_feature 1..20: note = Primer
  source 1..20: mol_type = other DNA
ccagaacctatgctggtgga

SEQ ID NO: 33 - DNA, 21 bp, synthetic construct
  misc_feature 1..21: note = Primer
  source 1..21: mol_type = other DNA
ggctttttggcttggttgact

SEQ ID NO: 34 - DNA, 21 bp, synthetic construct
  misc_feature 1..21: note = Primer
  source 1..21: mol_type = other DNA
aggaacaggtatcttggctct

SEQ ID NO: 35 - DNA, 20 bp, synthetic construct
  misc_feature 1..20: note = Primer
  source 1..20: mol_type = other DNA
ggggtgtagattggtgggaa

SEQ ID NO: 36 - DNA, 20 bp, synthetic construct
  misc_feature 1..20: note = Primer
  source 1..20: mol_type = other DNA
gtagtggaaaaccagcagcc

SEQ ID NO: 37 - DNA, 20 bp, synthetic construct
  misc_feature 1..20: note = Primer
  source 1..20: mol_type = other DNA
cctcctcgtcgcagtagaaa

SEQ ID NO: 38 - DNA, 22 bp, synthetic construct
  misc_feature 1..22: note = Primer
  source 1..22: mol_type = other DNA
tgcaaggatgacttggttacag

SEQ ID NO: 39 - DNA, 22 bp, synthetic construct
  misc_feature 1..22: note = Primer
  source 1..22: mol_type = other DNA
cttctgcaactgtgtctccaac

SEQ ID NO: 40 - DNA, 21 bp, synthetic construct
  misc_feature 1..21: note = Primer
  source 1..21: mol_type = other DNA
aagctccaggatgaaaccaac

SEQ ID NO: 41 - DNA, 21 bp, synthetic construct
  misc_feature 1..21: note = Primer
  source 1..21: mol_type = other DNA
agcgactcaatcttcctctcc

SEQ ID NO: 42 - DNA, 20 bp, synthetic construct
  misc_feature 1..20: note = Primer
  source 1..20: mol_type = other DNA
tggaaggactcatgaccaca

SEQ ID NO: 43 - DNA, 20 bp, synthetic construct
  misc_feature 1..20: note = Primer
  source 1..20: mol_type = other DNA
atgatgttctggagagcccc

SEQ ID NO: 44 - DNA, 20 bp, synthetic construct
  misc_feature 1..20: note = Primer
  source 1..20: mol_type = other DNA
ggccatcttttctgttgggg

SEQ ID NO: 45 - DNA, 19 bp, synthetic construct
  misc_feature 1..19: note = Primer
  source 1..19: mol_type = other DNA
tcagcattgaattccgccg

SEQ ID NO: 46 - DNA, 20 bp, synthetic construct
  misc_feature 1..20: note = Primer
  source 1..20: mol_type = other DNA
ctgaccctgcactcaatcaa

SEQ ID NO: 47 - DNA, 20 bp, synthetic construct
  misc_feature 1..20: note = Primer
  source 1..20: mol_type = other DNA
tccatcggaaggactaggtg

SEQ ID NO: 48 - DNA, 20 bp, synthetic construct
  misc_feature 1..20: note = Primer
  source 1..20: mol_type = other DNA
gtctcagtccagcacgtttg

SEQ ID NO: 49 - DNA, 20 bp, synthetic construct
  misc_feature 1..20: note = Primer
  source 1..20: mol_type = other DNA
gaaacgccgggaatactgtg

SEQ ID NO: 50 - DNA, 20 bp, synthetic construct
  misc_feature 1..20: note = Primer
  source 1..20: mol_type = other DNA
ccacatgggactttttgacc

SEQ ID NO: 51 - DNA, 20 bp, synthetic construct
  misc_feature 1..20: note = Primer
  source 1..20: mol_type = other DNA
ttactcggcgacagatttcc

SEQ ID NO: 52 - DNA, 19 bp, synthetic construct
  misc_feature 1..19: note = Primer
  source 1..19: mol_type = other DNA
ctttgggtgcgacttgacg

SEQ ID NO: 53 - DNA, 18 bp, synthetic construct
  misc_feature 1..18: note = Primer
  source 1..18: mol_type = other DNA
gtcgaccccgctcctttt

SEQ ID NO: 54 - DNA, 20 bp, synthetic construct
  misc_feature 1..20: note = Primer
  source 1..20: mol_type = other DNA
gcaggtggttgagagtgctt

SEQ ID NO: 55 - DNA, 20 bp, synthetic construct
  misc_feature 1..20: note = Primer
  source 1..20: mol_type = other DNA
gcacccgcctaagattcttc

SEQ ID NO: 56 - DNA, 21 bp, synthetic construct
  misc_feature 1..21: note = Primer
  source 1..21: mol_type = other DNA
tccaaaatcggatcaacagac

SEQ ID NO: 57 - DNA, 21 bp, synthetic construct
  misc_feature 1..21: note = Primer
  source 1..21: mol_type = other DNA
agagccacatttggatgtcac

SEQ ID NO: 58 - DNA, 23 bp, synthetic construct
  misc_feature 1..23: note = Primer
  source 1..23: mol_type = other DNA
tgggatttacaggcgtgagccac

SEQ ID NO: 59 - DNA, 24 bp, synthetic construct
  misc_feature 1..24: note = Primer
  source 1..24: mol_type = other DNA
aagcaaagcctcccaatcccaaac

SEQ ID NO: 60 - DNA, 20 bp, synthetic construct
  misc_feature 1..20: note = Primer
  source 1..20: mol_type = other DNA
agggatcaaagcctggaaca

SEQ ID NO: 61 - DNA, 20 bp, synthetic construct
  misc_feature 1..20: note = Primer
  source 1..20: mol_type = other DNA
ttggagcctgagacacgatt

SEQ ID NO: 62 - DNA, 21 bp, synthetic construct
  misc_feature 1..21: note = Primer
  source 1..21: mol_type = other DNA
gaaacagccatagagggcaaa

SEQ ID NO: 63 - DNA, 23 bp, synthetic construct
  misc_feature 1..23: note = Primer
  source 1..23: mol_type = other DNA
tggttttccagagtcttcagtga

SEQ ID NO: 64 - DNA, 22 bp, synthetic construct
  misc_feature 1..22: note = Primer
  source 1..22: mol_type = other DNA
gggctctcccatgcattcaaac

SEQ ID NO: 65 - DNA, 22 bp, synthetic construct
  misc_feature 1..22: note = Primer
  source 1..22: mol_type = other DNA
caccttccctccaaccagttgc

SEQ ID NO: 66 - DNA, 20 bp, synthetic construct
  misc_feature 1..20: note = Primer
  source 1..20: mol_type = other DNA
gccgagatctcagccatatt

SEQ ID NO: 67 - DNA, 20 bp, synthetic construct
  misc_feature 1..20: note = Primer
  source 1..20: mol_type = other DNA
atgtacttagaggtacaaat

SEQ ID NO: 68 - DNA, 20 bp, synthetic construct
  misc_feature 1..20: note = Primer
  source 1..20: mol_type = other DNA
tgacagaggaacgggggtat

SEQ ID NO: 69 - DNA, 20 bp, synthetic construct
  misc_feature 1..20: note = Primer
  source 1..20: mol_type = other DNA
cggctgtgatcaaatggcag

SEQ ID NO: 70 - DNA, 20 bp, synthetic construct
  misc_feature 1..20: note = Primer
  source 1..20: mol_type = other DNA
agtggttcttctgcgctact

SEQ ID NO: 71 - DNA, 21 bp, synthetic construct
  misc_feature 1..21: note = Primer
  source 1..21: mol_type = other DNA
ctgctggaaggtaaactctgg

SEQ ID NO: 72 - DNA, 23 bp, synthetic construct
  misc_feature 1..23: note = Primer
  source 1..23: mol_type = other DNA
acatacagtgattatttccccgt

SEQ ID NO: 73 - DNA, 20 bp, synthetic construct
  misc_feature 1..20: note = Primer
  source 1..20: mol_type = other DNA
cgccccaaagatgaggagta

SEQ ID NO: 74 - DNA, 20 bp, synthetic construct
  misc_feature 1..20: note = Primer
  source 1..20: mol_type = other DNA
aggagaggtccgaggaggtg

SEQ ID NO: 75 - DNA, 20 bp, synthetic construct
  misc_feature 1..20: note = Primer
  source 1..20: mol_type = other DNA
ctcagctccacctccgatag

SEQ ID NO: 76 - DNA, 21 bp, synthetic construct
  misc_feature 1..21: note = Primer
  source 1..21: mol_type = other DNA
ccatgcaggttgacaccgttg

SEQ ID NO: 77 - DNA, 21 bp, synthetic construct
  misc_feature 1..21: note = Primer
  source 1..21: mol_type = other DNA
tcggcagactgattcaaataa

SEQ ID NO: 78 - DNA, 21 bp, synthetic construct
  misc_feature 1..21: note = Primer
  source 1..21: mol_type = other DNA
tgaaatagcttagcggcaaga

SEQ ID NO: 79 - DNA, 21 bp, synthetic construct
  misc_feature 1..21: note = Primer
  source 1..21: mol_type = other DNA
gtgaatcgggaacagttgtgt

SEQ ID NO: 80 - DNA, 20 bp, synthetic construct
  misc_feature 1..20: note = Primer
  source 1..20: mol_type = other DNA
aagaggtggctgcttcaaac

SEQ ID NO: 81 - DNA, 20 bp, synthetic construct
  misc_feature 1..20: note = Primer
  source 1..20: mol_type = other DNA
agcctagattttcggccatc

SEQ ID NO: 82 - DNA, 20 bp, synthetic construct
  misc_feature 1..20: note = Primer
  source 1..20: mol_type = other DNA
atgaatcagtggatggcaca

SEQ ID NO: 83 - DNA, 20 bp, synthetic construct
  misc_feature 1..20: note = Primer
  source 1..20: mol_type = other DNA
ccattgagtgcctggatctt

SEQ ID NO: 84 - DNA, 20 bp, synthetic construct
  misc_feature 1..20: note = Primer
  source 1..20: mol_type = other DNA
ctcaggggcctttggacatc

SEQ ID NO: 85 - DNA, 20 bp, synthetic construct
  misc_feature 1..20: note = Primer
  source 1..20: mol_type = other DNA
caggcagtcgcagttttcac

SEQ ID NO: 86 - DNA, 20 bp, synthetic construct
  misc_feature 1..20: note = Primer
  source 1..20: mol_type = other DNA
tcttacgaggagctgcagac

SEQ ID NO: 87 - DNA, 20 bp, synthetic construct
  misc_feature 1..20: note = Primer
  source 1..20: mol_type = other DNA
tatccagctccagagtctct

SEQ ID NO: 88 - DNA, 20 bp, synthetic construct
  misc_feature 1..20: note = Primer
  source 1..20: mol_type = other DNA
ttcctgaggcacctgaagag

SEQ ID NO: 89 - DNA, 23 bp, synthetic construct
  misc_feature 1..23: note = Primer
  source 1..23: mol_type = other DNA
ggtgttccattttcatcatgacc

SEQ ID NO: 90 - DNA, 20 bp, synthetic construct
  misc_feature 1..20: note = Primer
  source 1..20: mol_type = other DNA
tagtgtgcccaaccatgagt

SEQ ID NO: 91 - DNA, 20 bp, synthetic construct
  misc_feature 1..20: note = Primer
  source 1..20: mol_type = other DNA
ttgcattcttcactggacca

SEQ ID NO: 92 - DNA, 20 bp, synthetic construct
  misc_feature 1..20: note = Primer
  source 1..20: mol_type = other DNA
aggttggctctgactgtacc

SEQ ID NO: 93 - DNA, 20 bp, synthetic construct
  misc_feature 1..20: note = Primer
  source 1..20: mol_type = other DNA
aaagctgttccgtcccagta

SEQ ID NO: 94 - RNA, 19 nt, synthetic construct
  misc_feature 1..19: note = Synthetic oligonucleotide
  source 1..19: mol_type = other RNA
taatctcggtgcctagtgt

(19) United States
(12) Patent Application Publication
(10) Pub. No.: US 2025/0101433 A1
(43) Pub. Date: Mar. 27, 2025

(54) MEANS AND METHODS FOR REDUCING TUMORIGENICITY OF CANCER STEM CELLS

(71) Applicants: B.G. NEGEV TECHNOLOGIES AND APPLICATIONS LTD., AT BEN-GURION UNIVERSITY, Beer Sheva (IL); THE NATIONAL INSTITUTE FOR BIOTECHNOLOGY IN THE NEGEV LTD., Beer Sheva (IL)

(72) Inventor: Varda Shoshan-Barmatz, Omer (IL)

(73) Assignees: B.G. NEGEV TECHNOLOGIES AND APPLICATIONS LTD., AT BEN-GURION UNIVERSITY, Beer Sheva (IL); THE NATIONAL INSTITUTE FOR BIOTECHNOLOGY IN THE NEGEV LTD., Beer Sheva (IL)

(21) Appl. No.: 18/976,183

(22) Filed: Dec. 10, 2024

Related U.S. Application Data
(62) Division of application No. 17/524,876, filed on Nov. 12, 2021.
Application No. 17/524,876 is a division of application No. 15/774,795, filed on May 9, 2018, now Pat. No. 11,174,484.
(60) Provisional application No. 62/253,142, filed on Nov. 10, 2015.

(51) Int. Cl. C12N 15/113; A61K 31/713; A61P 35/00; C12Q 1/68; C12Q 1/6886

(52) U.S. Cl. CPC C12N 15/1138; A61K 31/713; A61P 35/00; C12Q 1/68; C12Q 1/6886; C12N 2310/14; C12Q 2600/118; C12Q 2600/158

(57) ABSTRACT

The present invention discloses the use of RNA inhibitory molecules, particularly siRNA molecules silencing the expression of Voltage-Dependent Ion Chanel-1 (VDAC1) for the induction of cancer stem cell differentiation and reduction of TAMs abundance and tumor angiogenesis, thereby preventing tumor invention and recurrence.

### CROSS-REFERENCE TO RELATED APPLICATIONS

This application is a divisional application of U.S. application Ser. No. 15/774,795, filed on May 9, 2018, which is a 371 application of International Application No. PC/IL2016/051215, filed on Nov. 10, 2016, which claims priority to U.S. Application No. 62/253,142, filed on Nov. 10, 2015, which is incorporated herein by reference.

### SEQUENCE LISTING

The Sequence Listing submitted herewith is an ASCII text file (2021-11-12_Sequence_Listing.text, created on Nov. 12, 2021, 22030 bytes) via EFS-Web is hereby incorporated by reference.

### FIELD OF THE INVENTION

The present invention relates to means and methods for inhibiting the tumorigenicity of cancer and cancer stem cells. Particularly, the present invention discloses the use of RNA inhibitory molecules, particularly siRNAs silencing the expression of Voltage-Dependent Ion Chanel-1 (VDAC1) for the induction of cancer stem cell differentiation, reduction of tumor epithelial mesenchymal transition (EMT), tumor associated angiogenesis and tumor associated macrophages (TAMs) associated with tumor microenvironment, thereby preventing tumor progression, invasion and recurrence.

### BACKGROUND OF THE INVENTION

Accumulating evidence suggests that many cancers are maintained in a hierarchical organization of rare, slowly dividing tumor-initiating cells, also termed cancer stem cells; rapidly dividing amplifying cells (precursor cells); and differentiated tumor cells. Cancer stem cells (CSCs) are found within tumors or hematological cancers and possess characteristics normally associated with normal stem cells such as self-renewal and the ability to differentiate into multiple cell types. CSCs persist in tumors as a distinct population and give rise to the quasi (not finally) differentiated and differentiated cells that form the bulk of the tumor mass and phenotypically characterize the disease. These cells have been demonstrated to be highly tumorigenic and to be fundamentally responsible for cancer metastasis and cancer recurrence. There is mounting evidence that such cells exist in almost all tumor types, but their number is significantly lower compared to the number of the bulk cancer cells. CSCs are also often called tumor initiating cells, cancer stem-like cells, stem-like cancer cells, highly tumorigenic cells, or super malignant cells.

The cancer stem cells are generally slow-growing and transiently quiescent thus are not responsive to traditional anti-cancer therapies targeted to fast-growing cells. Therefore, traditional cancer therapies are likely to inhibit the bulk tumor population but not cancer stem cells, leaving the cancer stem cells intact and able to give rise to tumor re-growth. Consequently, cancer may recur as the result of cancer stem cell-driven expansion.

The existence of cancer stem cells has several implications in terms of cancer treatment and therapy. These include disease identification, selective drug targets, prevention of cancer metastasis and recurrence, treatment of cancer refractory to chemotherapy and/or radiotherapy, treatment of cancers inherently resistant to chemotherapy or radiotherapy and development of new strategies in fighting cancer.

Gliomas are the most common brain tumors, and, in particular, grade IV glioma, also named glioblastoma multiforme (GBM), represents the most severe type. This highly aggressive tumor is associated with high rates of morbidity, relapse and mortality (Bai RY et al., 2011. Trends in molecular medicine 17, 301-312). GBM is a heterogeneous cancer, with tumors containing niches enriched for transiently quiescent and self-renewing cells that are essentially GBM cancer stem cells (GSCs) (Chen J et al., 2012. Nature 488, 522-526). Such intra-tumoral heterogeneity underlies the inability of conventional and targeted therapies to achieve long-term remission (Nathanson D A et al., 2014. Science 343, 72-76.; Sottoriva A et al., 2013. PNAS USA 110, 4009-4014).

Cancer cells acquire a common set of properties, including high proliferation, resistance to apoptosis and metabolic reprogramming, such as enhanced anaerobic glycolysis (Warburg effect; Hanahan, D, and Weinberg, R A 2011. Cell 144, 646-674; Koppenol W H et al. 2011. Nature reviews Cancer 11, 325-337). As such, cancer cells up-regulate the transcription of genes related to glycolysis. Mitochondria play a role in reprogramming cellular metabolism, with metabolic flexibility serving to balance tumor cell energy needs with requirements for metabolites and precursors. Metabolic reprogramming in GBM and its influence on epigenetics were recently reviewed (Agnihotri. S and Zadeh G 2016. Neuro Oncol 18, 160-172).

Discovery of CSCs in glioblastoma as well as in other cancer types has opened a new target for treatment. For example, U.S. Pat. No. 7,608,259 discloses improved methods for treatment of cancer which involve the targeting of slow-growing, relatively mutationally-spared cancer stem line

U.S. Pat. No. 8,129,184 discloses methods and compositions for cancer vaccines that target cancer stem cells. Particularly the invention discloses stimulating the immune response by administering to a patient a composition including dendritic cells that present cancer stem cell antigens. The method is used for treating cancer, glioblastoma being a specific embodiment.

U.S. Pat. No. 8,846,633 discloses a method for inhibiting cancer stem cell like properties and chemo- radio-resistant properties of cancer or tumor cells comprising delivering miR145 to the cancer or tumor cells, particularly brain tumor and head and neck cancer cells. The invention further provides a pharmaceutical composition comprising miR145 and a method for treating brain tumor and/or head and neck cancer comprising administration of miR145 to a subject in need thereof.

U.S. Pat. No. 9,078,857 discloses compositions comprising inhibitors of Ephrin (EPH) receptor expression in tumor stem cells and methods of use thereof for treating cancer.

U.S. Patent Application Publication No. 2013/0209413 discloses use of a parvovirus, preferably H-1PV, for the therapeutic elimination of cancer stem cells (CSCs), preferably neuroblastoma stem cells and glioblastoma stem cells.

U.S. Patent Application Publication No. 2014/0377263 discloses a method of treating and/or preventing cancer comprising targeting cancer stem cells by administering miRNAs which have reduced expression or are lacking in the cancer stem cells. In some embodiments, the miRNAs that are reduced or lacking in cancer stem cells are let-7 miRNAs. In alternative embodiments, the invention discloses a method of treating and/or preventing cancer comprising targeting cancer stem cells by administering miRNAs which have increased expression levels in the cancer stem cells.

Voltage-dependent anion channel 1 (VDAC1) is a mitochondrial protein controlling cell energy and metabolic homeostasis (Shoshan-Barmatz V et al., 2015; Biochim. Biophys. Acta 1848, 2547-2575; Shoshan-Barmatz V et al., 2010. Molecular aspects of medicine 31, 227-285). VDAC1 is the sole channel located at the outer mitochondrial membrane (OMM) mediating metabolic cross-talk between mitochondria and the cytosol, transporting metabolites, ions, nucleotides, Ca²⁺ and more, thus regulating mitochondrial activity. VDAC1 also plays a key role in apoptosis, participating in the release of apoptotic factors from mitochondria and interacting with anti-apoptotic regulators (Shoshan-Barmatz et al., 2015, ibid; Shoshan-Barmatz et al., 2010, ibid). VDAC1 is also highly expressed in different tumors (Shoshan-Barmatz et al., 2015 ibid), including astrocytic tumors (Feichtinger R G et al., 2014. Glia 62, 514-525), pointing to its significance in high energy-demanding cancer cells.

An inventor of the present invention and co-worker have demonstrated that abrogation of VDAC1 expression by 2′-O-Me-modified siRNA specific to human (h)VDAC1 reduced cellular ATP levels and cell growth and inhibited solid tumor development and growth in cervical and lung cancers (U.S. Pat. No. 8,093,369; Koren I et al., 2010 Cancer biology & therapy 9, 1046-1052; Arif T et al., 2014. Molecular therapy Nucleic acids 3, e159). As a key regulator of metabolic and energy reprogramming, disrupting cancer energy and metabolism homeostasis by targeting VDAC1 offers a potential anti-cancer therapy strategy (Maldonado E N and Lemasters J J. 2012. J Pharmacol Exp her 342, 637-641).

A paper of the present inventor and co-workers published after the priority date of the present invention descries that VDAC1 deletion led to up-regulation of pro-apoptotic proteins including caspases, p53 and Cytochrome c, yet without apoptosis induction. Rather, this up-regulation was associated with cell differentiation and down regulation of SMAC/Diablo, AIF and translocator protein (TSPO), all connected to cell growth support. (Arif T et al. 2016. Biochim Biophys Acta 1857(8), 1228-42). The results show that the activity of apoptotic proteins extends beyond the realm of cell death induction, revealing roles at the intersection between oncogene-induced changes in metabolism and the expression of pro-apoptotic proteins, to the benefit of the cancer cell.

Current approaches for tumor treatment many times do not prevent recurrence, particularly in certain cancer types including, for example, glioblastoma. Tumor recurrence is a major cause of mortality; thus, there is a great need for and it would be highly advantageous to have means to reduce tumorigenicity and cancer recurrence.

### SUMMARY OF THE INVENTION

The present invention relates to the reduction of tumorigenicity of tumors comprising cancer stem cells, particularly to the prevention of the tumor recurrence, tumor cancer stem cell (CSC) invasion, tumor angiogenesis and tumor-favorable microenvironment, and to induction of tumor cancer stem cells differentiation leading to reduction in CSC.

The present invention is based in part on the unexpected discovery that silencing the expression of VDAC1 in human glioblastoma tumor cells had led to reprogramming of the glioblastoma stem cells towards differentiation into non-replicating end-stage mature-like neurons and astrocytes. Several markers associated with cancer stem cells were disappeared while markers of differentiated astrocytes and neuron-like cells appeared. The present invention now discloses hitherto unknown effects of silencing the expression of VDAC1 on several cancer-associated biological processes, including induction of cancer stem cells differentiation, reversing the epithelial to mesenchymal transition (EMT), reversing the reprogrammed cancer cell metabolism towards metabolism of healthy cells, reduced TAMs abundance and NF-kB expression as a pro-inflammatory marker and inhibition of angiogenesis.

Thus, the invention now provides means and methods for treating cancers that are characterized by the presence of cancer stem cells, particularly for preventing the recurrence of such cancers, for reducing tumor invasiveness and for reducing angiogenesis utilizing VDAC1-silencing oligonucleotides or recombinant constructs encoding same.

According to one aspect, the present invention provides a method of reducing the tumorigenicity of a tumor, the method comprises administering to, or expressing in cells of a subject affected with a tumor comprising cancer stem cells (CSCs) an effective amount of at least one voltage dependent anion channel 1 (VDAC1)-silencing oligonucleotide, thereby reducing the number and/or frequency of the CSCs in said tumor.

According to certain embodiments, the tumor is a solid tumor. According to some embodiments, the solid tumor is selected from the group consisting of brain, breast, prostate, cervical, ovary, pancreas, head and neck, sarcoma, lymphoma, melanoma and colon cancer. Each possibility represents a separate embodiment of the present invention. According to certain embodiments, the brain tumor is glioma. According to certain embodiments, the glioma is selected from the group consisting of astrocytoma, oligodendroglioma, and glioblastoma. According to certain exemplary embodiments, the glioma is glioblastoma multiforme (GBM; glioma stage IV).

According to certain embodiments, the tumor is a non-solid tumor. According to some embodiments, the non-solid tumor is blood cancer.

According to certain embodiments, the VDAC1-silencing oligonucleotide comprises at least 15 contiguous nucleic acids identical to the gene or mRNA encoding human VDAC1 protein or to a complementary polynucleotide thereof, wherein the human VDAC1 protein comprises the amino acid sequence set forth in SEQ ID NO:1.

According to certain embodiments the hVDAC1 protein is encoded by the nucleic acid sequence set forth in SEQ ID NO:2.

Any silencing oligonucleotide molecule as is known in the art can be used according to the teachings of the present invention as long as the oligonucleotide comprises at least 15 contiguous nucleic acids identical to SEQ ID NO:2, to an mRNA molecule encoded by same or to a sequence complementary thereto.

According to certain embodiments, the silencing oligonucleotide comprises a nucleic acid sequence selected from the group consisting of SEQ ID NO:3 (ACACUAGGCACCGAGAUUA); SEQ ID NO:4 (GGGCUAUGGAUUUGGCUUA); SEQ ID NO:5 (GCUUGGUCUAGGACUGGAA); SEQ ID NO:6 (AAGCUGACCUUCGAUUCAU); SEQ ID NO:7 (GAAUGACGGGACAGAGUUU); SEQ ID NO:8 (UCGGAAUAGCAGCCAAGUA); SEQ ID NO:9 (CUCUUCUGGAUGGCAAGAA); SEQ ID NO: 10 (GAAUAGCAGCCAAGUAUCAG) and a complementary sequence thereto.

According to certain embodiments, the VDAC1-silencing oligonucleotide is selected from the group consisting of RNA interference (RNAi) molecule and antisense molecule. According to some embodiments, the RNAi molecule is an unmodified and/or modified double stranded (ds) RNA molecules including, but not limited to, short-temporal RNA (stRNA), small interfering RNA (siRNA), short-hairpin RNA (shRNA), and microRNA (miRNA).

According to certain exemplary embodiments, the RNAi is siRNA.

According to some exemplary embodiments, the siRNA comprises a first oligonucleotide sequence identical to at least 15 nucleotides of SEQ ID NO:2 or to a mRNA encoded by same and a second oligonucleotide sequence substantially complementary to the first oligonucleotide; wherein said first and second oligonucleotide sequences are annealed to each other to form the siRNA molecule.

According to some embodiments, the siRNA is a single-stranded short hairpin RNA (shRNA) wherein the first oligonucleotide sequence is separated from the second oligonucleotide sequence by a linker which forms a loop structure upon annealing of the first and second oligonucleotide sequences. In some embodiments the linker is about 3 to about 60 nucleotides.

According to some exemplary embodiments, the siRNA comprises a first oligonucleotide having the nucleic acid sequence set forth in SEQ ID NO:3 (ACACUAGGCACCGAGAUUA) and a second oligonucleotide having the nucleic acid sequence set forth in SEQ ID NO:94 (UAAUCUCGGUGCCUAGUGU).

According to some exemplary embodiments, the siRNA comprises a first oligonucleotide having the nucleic acid sequence set forth in SEQ ID NO:4 (GGGCUAUGGAUUUGGCUUA) and a second oligonucleotide having the nucleic acid sequence set forth in SEQ ID NO:11 (UAAGCCAAAUCCAUAGCCC).

According to some exemplary embodiments, the siRNA comprises a first oligonucleotide having the nucleic acid sequence set forth in SEQ ID NO:5 (GCUUGGUCUAGGACUGGAA) and a second oligonucleotide having the nucleic acid sequence set forth in SEQ ID NO:12 (UUCCAGUCCUAGACCAAGC).

According to some exemplary embodiments, the siRNA comprises a first oligonucleotide having the nucleic acid sequence set forth in SEQ ID NO: 10 (GAAUAGCAGCCAAGUAUCAG) and a second oligonucleotide having the nucleic acid sequence set forth in SEQ ID NO:13 (UGAUACUUGGCUGCUAUUC).

According to additional embodiments, at least one of the siRNA nucleic acids is chemically modified. Typically, the modification is 2′-O-methyl modification of a guanine or uracil. According to certain embodiments, the first and the second polynucleotide of the RNAi comprise several chemically modified guanine and/or uracil nucleotides. According to certain exemplary embodiments, the modified siRNA molecule comprises a first oligonucleotide having the nucleic acid sequence set forth in SEQ ID NO:14 (ACACUAGGCACCGAGAUUA), wherein the Uracil and Guanine nucleotides marked in bold and underline comprise 2′-O-methyl modification and a second oligonucleotide having the nucleic acid sequence set forth in SEQ ID NO:15 (UAAUCUCGGUGCCUAGUGU, wherein the Uracil and Guanine nucleotides marked in bold and underline comprise 2′-O-methyl modification.

According to certain embodiments, the method comprises administering to the subject an isolated oligonucleotide silencing the expression of VDAC1. According to some embodiments, the method comprises administering to the subject an isolated oligonucleotide comprising a nucleic acid sequence selected from the group consisting of SEQ ID NOs:3-10. Each possibility represents a separate embodiment of the present invention. According to some embodiment, the method comprises administering to the subject an isolated siRNA molecule comprising the nucleic acid sequence set forth in any one of SEQ ID NOs:11-15 and 94. According to certain exemplary embodiments, the method comprises administrating to the subject an isolated siRNA oligonucleotide comprises a first oligonucleotide having the nucleic acid sequence set forth in SEQ ID NO:3 and a second oligonucleotide having the nucleic acid sequence set forth in SEQ ID NO:94.

According to certain exemplary embodiments, the method comprises administrating to the subject an isolated siRNA oligonucleotide comprises a first oligonucleotide having the nucleic acid sequence set forth in SEQ ID NO:14 and a second oligonucleotide having the nucleic acid sequence set forth in SEQ ID NO: 15.

According to certain embodiments, the method comprises administering to the subject a construct capable of expressing in cells of said subject a therapeutically effective amount of at least one VDAC1-silencing oligonucleotide. According to some embodiments, the method comprises administering to the subject a construct capable of expressing at least one oligonucleotide comprising a nucleic acid sequence selected from the group consisting of SEQ ID Nos:3-10. According to some embodiment, the method comprises administering to the subject a construct capable of expressing siRNA molecule comprising the nucleic acid sequence set forth in any one of SEQ ID Nos:11-15 and 94. According to certain exemplary embodiments, the method comprises administrating to the subject a construct capable of expressing siRNA oligonucleotide comprises a first oligonucleotide having the nucleic acid sequence set forth in SEQ ID NO:3 and a second oligonucleotide having the nucleic acid sequence set forth in SEQ ID NO:94. According to certain exemplary embodiments, the method comprises administrating to the subject a construct capable of expressing siRNA oligonucleotide comprises a first oligonucleotide having the nucleic acid sequence set forth in SEQ ID NO:14 and a second oligonucleotide having the nucleic acid sequence set forth in SEQ ID NO:15.

According to certain embodiments, reducing the tumorigenicity of the tumor comprises attenuating the invasive potential of said tumor, particularly of the tumor cancer stem cells. Without wishing to be bound by any specific theory or mechanism of action, reduction in the invasive potential of the tumor cells and/or the tumor cancer stem cells is attributed, inter alia, to the reversal of the epithelial to mesenchymal transition (EMT) process.

According to certain embodiments, reducing the tumorigenicity of the tumor comprises reducing the recurrence of said tumor.

According to additional aspect, the present invention provides a method for inducing the transition of a cancer stem cell to a differentiated cell, the method comprises administrating to or expressing in the cancer stem cell an effective amount of at least one voltage dependent anion channel 1 (VDAC1)-silencing oligonucleotide.

According to certain embodiments, the cancer stem cells are isolated cells present in a cell culture. According to other embodiments, the cancer stem cells are present within a tumor in a subject.

The VDAC1-silencing oligonucleotide and constructs comprising same are as described hereinabove.

As described in details in the examples section hereinbelow, the present invention discloses for the first time that silencing the expression of hVDAC1 by siRNA molecules had a significant impact on the expression of glioblastoma stem cells markers.

Thus, according to another aspect, the present invention provides a method for monitoring potential recurrence of glioblastoma in a subject, the method comprises determining the level of at least one marker selected from the group consisting of CD133, c-kit, Sox2, Klf4, Oct4, Nanog, Musashi, Nestin, NGFR, S100b, CD44 and any combination thereof in a sample comprising glioblastoma cancer cells obtained from the subject, wherein a level above a predetermined reference level correlates with a high potential of glioblastoma recurrence in said subject.

According to certain embodiments, the predetermined reference level is determined in a sample comprising corresponding glioblastoma cancer cells treated with an effective amount of VDAC1-silencing oligonucleotide molecule.

According to certain embodiments, the reference level is determined in a sample comprising glioblastoma cancer cells obtained from a tumor treated with the VDAC1-silencing molecule. According to other embodiments, the reference level is determined in a sample comprising cultured glioblastoma cancer cells treated with the VDAC1-silencing molecule. According to yet additional embodiments, the reference level is an average level of the at least one marker determined in samples obtained from healthy subjects.

The VDAC1 silencing oligonucleotide molecule is as described hereinabove.

According to yet additional aspect, the present invention provides a method for reducing angiogenesis in a tumor tissue, the method comprises administering to, or expressing in the tissue an effective amount of voltage dependent anion channel 1 (VDAC1)-silencing oligonucleotide molecule. The VDAC1 silencing oligonucleotide molecule and constructs comprising same are as described hereinabove.

According to yet additional aspect, the present invention provides a method for reducing tumor associated macrophages (TAMS) abundance in a tumor tissue, the method comprises administering to, or expressing in the tissue an effective amount of voltage dependent anion channel 1 (VDAC1)-silencing oligonucleotide molecule. The VDAC1 silencing oligonucleotide molecule or constructs comprising same are as described hereinabove.

According to yet additional aspect, the present invention discloses voltage dependent anion channel 1 (VDAC1)-silencing oligonucleotide or a construct comprising same for use in reducing the tumorigenicity of a tumor, wherein the tumor comprises cancer stem cells.

Any method as is known in the art for the administration of VDAC1 silencing oligonucleotide molecule, particularly VDAC1-directed siRNA or a construct comprising same can be used according to the teachings of the present invention.

According to certain embodiments, the VDAC1 silencing oligonucleotide molecule or a construct comprising same is administered within a pharmaceutical composition. According to certain exemplary embodiments, the pharmaceutical composition further comprises pharmaceutically acceptable excipients, diluents or carriers. According to additional exemplary embodiments, the VDAC1 silencing oligonucleotide molecule is encapsulated within a nanoparticle or a liposome. According to some embodiments, the VDAC1 silencing oligonucleotide molecule is encapsulated within Polyethylenimine (PEI)-Poly(D,L-lactide-co-glycolide) (PLGA) nanoparticle.

According to some embodiments, the VDAC1 silencing oligonucleotide molecule, construct comprising same or a composition comprising same is administered via intravenous, intradermal, intramuscular, intraarterial, intralesional, percutaneous, subcutaneous, intranasal or inhalation or by aerosol administration, or combinations thereof. In some embodiments, administration is prophylactic administration, and in alternative embodiments, administration is therapeutic administration.

According to certain embodiments, the subject is a mammal, particularly human. According to some embodiments, the subject has previously undergone at least one or more cancer therapies including, but not limited to, surgery, chemotherapy, radiotherapy, thermotherapy, immunotherapy, hormone therapy and laser therapy.

Other objects, features and advantages of the present invention will become clear from the following description and drawings.

### BRIEF DESCRIPTION OF THE FIGURES

FIGS. 1A-1I show morphological changes and expression of neuronal markers associated with cell differentiation in GBM. Human normal (FIG. 1A) or GBM brains (FIG. 1B) stained with H&E or anti-GFAP antibodies. Human VDAC1 silencing siRNA (si-hVDAC1) (FIG. 1C) or scrambled siRNA (si-Scr) (FIG. 1D) treated U-87MG xenograft tumors sections stained with H&E or anti-GFAP antibodies. FIG. 1E-Typical sections from si-Scr- and si-hVDAC1-treated tumor (TT)s, immune-stained for Map2 and TUBB3. FIG. 1F: WB of GFAP and TUBB3 in si-Scr- and si-hVDAC1-TTs. RU=relative units. FIG. 1G: Schematic presentation of early precursor cell differentiation into mature astrocytes via several possible intermediate states: Late precursor cells, immature astrocytes, or neurons via immature neurons. mRNA levels of markers specific for each state in si-hVDAC1-TTs relative to those in si-Scr-TTs are presented. Results are the mean±SEM (n=3-5 tumors); p:*≤0.05; **≤0.001; ***≤0.0001). FIG. 1H: Immunofluorescent staining of si-Scr- and si-hVDAC1-TTs-derived sections for Nestin, GFAP, TUBB3 and GAD-67. FIG. 1I: IHC staining of VDAC1 of human normal brain (n=13) or glioblastoma (GBM) (n=41) in tissue microarray slides (Biomax). Percentages of sections stained at the intensity indicated are shown.

FIGS. 2A-2C show increased expression of differentiation markers in si-hVDAC1-TTs. FIG. 2A. Proteins extracted from si-Scr- or si-hVDAC1-TTs derived from U-118MG cells were immunoblotted for TUBB3, GFAP and GAD-67. β-actin immunostaining serving as a loading control is presented. RU=relative unit. FIG. 2B: IHC staining of si-Scr- or si-hVDAC1-TT sections from a U-118MG-derived tumor using specific antibodies against TUBB3, GFAP and GAD-67. FIG. 2C: The area circled by black lines indicates cells populations organized in clusters that may point to group of GSCs that undergo differentiation process.

FIGS. 3A-3L show alteration of transcription factors (TF) levels and stem cells and EMT markers expression in si-hVDAC1-TTs. FIG. 3A shows the change in the expression of stem cells TFs as analyzed using specific kit. FIG. 3B: qRT-PCR analysis of p53, c-Myc and Hif-1α, mRNA levels in U-87MG si-hVDAC1-TTs, relative to si-Scr-TTs. Results mean±SEM (n=3-5 tumors); p: *≤0.05; **≤0.001. FIG. 3C-D: Western blotting (WB) of p53 in si-Scr- and si-hVDAC1-TTs derived from U-87MG (FIG. 3C) orU-118MG cells (FIG. 3D). FIG. 3E-F: IHC staining of si-Scr- or si-hVDAC1-TTs sections for stem cell markers c-Kit, Klf4, Sox2, Musashi, Nestin, NGFR, S100b, CD44. FIG. 3G: WB of CD133. Klf4, NGFR and S100b in si-Scr- and si-hVDAC1-TTs. FIG. 3H: qRT-PCR analysis of Sox2. Oct3/4 and Nanog mRNA. Results=mean±SEM (n=3-5 tumors), p: *≤0.05; **≤0.001. FIG. 3I: Orthotopic mouse model, representative Nestin-stained sections from brains engrafted with si-Scr- or si-VDAC1-treated U-87MG cells, 22 days after cell grafting. FIG. 3J: Typical IHC staining of sections from si-Scr- or si-hVDAC1-TTs for E-cadherin, N-cadherin and Vimentin. FIG. 3K: WB of E-cadherin and vimentin in U-87MG si-Scr- and si-hVDAC1-TTs. FIG. 3L: qRT-PCR analysis of Twist, Zeb1, Zeb2, Snail1, Snail2 and N-cadherin (N-Cadh) mRNA levels in si-Scr- and si-hVDAC1-TTs. Results are the mean±SEM (n=3-5, p: *≤0.05; **≤0.001). RU=average relative levels.

FIGS. 4A-4E show inhibition of invasion in si-hVDAC1-TTs with altered expression of stem cell markers. FIG. 4A: H&E staining of si-Scr- and si-hVDAC1-treated U-87MG and U-118 tumors, showing representative sections. Arrows point to muscle, indicting of tumor invasion. FIG. 4B: Representative IHC staining for Nestin of sections from brains engrafted with si-Scr- or si-VDAC1-treated U-87MG cells, 22 days after cells engraftation. Arrows point to tumor invasion in the brain. FIG. 4C: IHC of si-Scr- or si-hVDAC1-TT sections from U-118 tumors stained for stem cell markers, Nestin, Sox2 and Klf4. FIG. 4D: WB of CD133, Klf4, CD44 and Nestin levels in si-Scr- and si-hVDAC1-TTs sections from U118-derived tumors RU=average relative levels. FIG. 4E: IHC staining of tumor sections derived from U-87MG tumors treated with si-Scr- or si-hVDAC1 using anti-F4/80, tumor associate macrophages marker.

FIGS. 5A-5D demonstrate inhibition of angiogenesis in si-hVDAC1-TTs with altered expression of endothelial cell markers CD31. FIG. 5A: Representative IHC staining of endothelial cells associated with blood vessels in sections from si-Ser- and si-hVDAC1-TTs derived from U-87MG tumors, as revealed using anti-CD31 antibodies. Black arrows point to blood vesicles (bar=50 μm). FIG. 5B: Quantitative analysis of microvessel density (MVD) per unit area expressed as means±SEM, p<0.001, si-Scr versus si-hVDAC1 (n=5 animals from each group). FIG. 5C, D: Immunoblotting (FIG. 5C) and IHC staining (FIG. 5D) of tumor sections derived from U-87MG tumors treated with si-Scr- or si-hVDAC1 using anti-VEGF antibodies.

FIGS. 6A-6E demonstrate DNA microarray and bioinformatics analyses of si-hVDAC1- and si-Scr-TTs FIG. 6A: Clustering of the 4,493 differentially expressed genes: Down-(cluster 1, 1998 genes) and up-regulated (cluster 2, 2495 genes). Black to gray colors indicates expression level. Promoter analysis indicate binding sites enrichment for FOXP1 (367 genes, p 3.4×10⁻²⁰) in cluster 1, and NRSF (1,310 genes, p=1.3×10⁻⁸⁴), HNF4 (1,161 genes, p=5.8×10⁻³⁸) and MAZ (678 genes, p=1.2×10⁻²²) in cluster 2. FIG. 6B: Functional analysis of cluster 1 and 2 based on the gene ontology (GO) system. The number of genes associated with the indicated function is presented. FIG. 6C: WB of HNF4 and FOXP1 in si-Scr- and si-hV DAC1-TTs. RU=Average relative level. FIG. 6D: IHC staining of si-Scr- or si-hVDAC1-TTs using antibodies specific to the TFs identified in A: FOXP1, NRSF, HNF4 and MAZ. FIG. 6E: Levels of Sox10, DLST, CAST, Selug2 and PTEN12 levels in si-Scr- and si-hVDAC1-TFs, as analyzed using qRT-PCR (Black bars) and DNA microarray (grey bars). Results are the mean±SEM (n=3-5, p: *≤0.05; **≤0.001).

FIGS. 7A-7L demonstrate si-hVDAC1 inhibition of tumor growth in vivo. FIG. 7A: U-87MG cells were s.c. inoculated into athymic nude mice. On day 13, the mice were divided into 2 groups and xenografts were injected every three days with si-Scr (●, 8 mice) or si-hVDAC1 (▴, 16 mice) to a final concentration of 50-60 nM (***p≤0.0001). FIG. 7B: On day 33, the si-hVDAC1-treated mice were sub-divided into 2 groups (8 mice each). One group (▴) continued si-hVDAC1 treatment, the other switched to si-Scr (◯) treatment. (●) indicates original si-Ser group. FIG. 7C: Representative sections from Scr- and si-hVDAC1-TTs IHC-stained for VDAC1. FIG. 7D: WB of VDAC1 in si-Scr- and si-hVDAC1-TTs. RU=relative units. FIG. 7 E, F: IHC of si-Scr- and si-hVDAC1-TTs stained for Ki-67 (FIG. 7E): positive cells counted over several fields (FIG. 7F). FIG. 7G: qRT-PCR analysis of Ki-67 and PCNA mRNA levels isolated from si-Scr- (black bars) and si-hVDAC1 (gray bars)-TTs. Results=mean±SEM (n=3-5), p: **≤0.001; ***≤0.001). FIG. 7 H, 1: IHC and WB analyses of EGFR. FIG. 7J: Orthotopic GBM. MRI imaging of brains 33 days after engrafted with U-87MG cells treated with si-Scr or si-VDAC1. FIG. 7K Calculated tumor volume after 22 (black bars) and 33 days (grey bars). Results=mean±SEM (n=4-5), p: ****≤e0.00001. FIG. 7L. Sections of paraffin-embedded U-87MG xenograft from si-Scr- or si-hVDAC1-TTs stained with PI or TUNEL showing no cell death.

FIGS. 8A-8G demonstrate reversal of the U-87MG tumor cell reprogrammed metabolism by si-hVDAC1 treatment. FIG. 8A-C shows IHC staining of si-Scr- or si-hVDAC1-TT sections using specific antibodies against Glut1. HK-I and HK-II; (FIG. 8A); GAPDH. LDH and VDAC1 (FIG. 8B); and CS, complex IVc and ATP synthase 5a (FIG. 8C). FIG. 8D, E shows Western blot (WB) of selected proteins RU=relative unit. FIG. 8F shows the decrease in mRNA levels of metabolic enzymes in si-hVDAC1-TTs relative to those in si-Scr-TTs are presented. Results=mean±SEM (n=3-5 tumors), p: *≤0.05; **≤001. FIG. 8G shows representative IHC sections from brains engrafted with si-Scr- or si-VDAC1-treated U-87MG cells, 22 days after cell grafting, stained for Glut 1 and VDAC1.

FIGS. 9A-9C show the effect of si-hVDAC1 and si-Scr on neurosphere formation. G7, MZ-18 and MZ327 cells were treated with si-Scr or si-hVDAC1 and after 48 and 72 h, the cells were analyzed for VDAC1 levels (FIG. 9A) or cell growth using SRB (FIG. 9B), as described in the Examples section hereinbelow. Results are the mean±SEM (n=3); p:**≤0.001; ***≤0.0001. FIG. 9C shows Neurosphere formation in si-Scr- or si-hVDAC1-treated U-87MG, G7, MZ-18 and MZ-327 cells in stem cell-specific medium.

### DETAILED DESCRIPTION OF THE INVENTION

The present invention discloses hitherto unknown outcomes resulting from reducing the expression of VDAC1 in cancer cell, particularly in cancer stem cells and quasi (not finally) differentiated cancer cells. Unexpectedly, the present invention shows that reduced expression of VDAC1, exemplified b % siRNA silencing of its encoding gene, has led to the differentiation of glioblastoma cancer stem cells into non-replicating end-stage-like neurons and astrocytes. It was further shown that the silencing had reversed EMT and reduced TAMs abundance and angiogenesis in the treated tumor tissues. Taken together, these unexpected phenomena of VDAC1 reduced expression can be used to reduce the tumorigenicity of tumors comprising cancer stem cells and to prevent recurrence of cancer diseases characterized by cancer-stem cells containing tumors, including, but not limited to brain, breast, prostate, cervical, ovary, pancreas, head and neck, sarcoma, lymphoma, melanoma and colon cancer. In certain exemplary embodiments, the brain cancer is glioblastoma.

### Definitions

The terms “VDAC1” and “hVDAC1” are used herein interchangeably and refer to the human voltage-depended anion channel isoform 1 (hVDAC1) of a highly conserved family of mitochondrial porin. Four VDAC isoforms, encoded by three genes, are known to date; as used herein, the terms “VDAC1” and “hVDAC1” refer to a 283 amino acid protein (NP_003365) having the amino acids sequence set forth in SEQ ID NO:1, encoded by a polynucleotide having the nucleic acid sequence set forth in SEQ ID NO:2.

As used herein, the term “cancer stem cells” refers to an undifferentiated cell which is capable of proliferation and giving rise to more progenitor cells having the ability to generate a large number of mother cells that can in turn give rise to differentiated or differentiable daughter cells. The daughter cells themselves can be induced to proliferate and produce progeny that subsequently differentiate into one or more mature cell types, while also retaining one or more cells with parental developmental potential. The term “cancer stem cell” refers then, to a cell with the capacity or potential, under particular circumstances, to differentiate to a more specialized or differentiated phenotype, and which retains the capacity, under certain circumstances, to proliferate without substantially differentiating. In one embodiment, the term progenitor or stem cell refers to a generalized mother cell whose descendants (progeny) specialize, often in different directions, by differentiation, e g., by acquiring completely individual characters, as occurs in progressive diversification of embryonic cells and tissues. Cellular differentiation is a complex process typically occurring through many cell divisions. A differentiated cell can derive from a multipotent cell which itself is derived from a pluripotent cell, and so on. While each of these multipotent cells can be considered stem cells, the range of cell types each can give rise to can vary considerably. Some differentiated cells also have the capacity to give rise to cells of greater developmental potential. Such capacity can be natural or can be induced artificially upon treatment with various factors. In many biological instances, stem cells are also “multipotent” because they can produce progeny of more than one distinct cell type, but this is not required for “stemness.” Self-renewal is the other classical part of the stem cell definition, and it is essential as used herein. Cancer stem cells have the ability for self-renewal, multipotent differentiation and vigorous proliferative capacity.

With reference to cells, the terms ““differentiated” or “differentiating” are relative terms; a “differentiated cell” is a cell that has progressed further down the developmental pathway with increased functional potential than the cell it is being compared with. Thus, stem cells can differentiate to lineage-restricted precursor cells (such as a mesodermal stem cell), which in turn can differentiate into other types of precursor cells further down the pathway, and then to an end-stage differentiated cell, which plays a characteristic role in a certain tissue type (for example neurons), and can or cannot retain the capacity to proliferate further.

As used herein “tumorigenic” refers to the functional features of a solid or non-solid tumor stem cell including the properties of self-renewal (giving rise to additional tumorigenic cancer stem cells) and proliferation to generate all other tumor cells (giving rise to differentiated and thus non-tumorigenic tumor cells) that allow cancer stem cells to form a tumor. The terms “reducing tumorigenicity” and “reduction of tumorigenicity” are used interchangeably herein and refer to the reduction in the number and/or frequency of cancer stem cells and cancer cells in a tumor leading to reduction in the tumor invasiveness and/or to the prevention of the tumor recurrence.

The term “stemness” as used herein refers to a cell with stem cell properties, for example a cell that has the capacity for self-renewal, for example a cell that is totipotent, pluripotent or multipotent. A cancer cell that is a “cancer stem cell” or a cancer cell with stemness properties is a cancer cell which can give rises to daughter cells which themselves can be induced to proliferate and produce progeny that subsequently differentiate into one or more mature cell types, while also retaining one or more cells with parental developmental potential.

The terms “oligonucleotide”, “oligonucleotide sequence”, “nucleic acid sequence”, and “polynucleotide” are used interchangeably herein and refer to an oligomer or polymer of ribonucleic acid (ribo-oligonucleotide or ribo-oligonucleoside) or deoxyribonucleic acid comprising up to about 100-1,000 nucleic acid residues. These terms encompass nucleotide sequences strands composed of naturally-occurring nucleobases, sugars and covalent inter-sugar linkages as well as oligonucleotides having non-naturally-occurring portions which function similarly. Such modified or substituted oligonucleotides may be preferred over native forms because of the valuable characteristics including, for example, increased stability in the presence of plasma nucleases and enhanced cellular uptake. An oligonucleotide may be a polymer of RNA or DNA or hybrid thereof, that is single- or double-stranded, linear or branched, and that optionally contains synthetic, non-natural or altered nucleotide bases. The terms also encompass RNA/DNA hybrids. It is to be explicitly understood that the oligonucleotide sequences provided herein can be of DNA or RNA molecules.

The terms “construct”, or “RNAi expression construct” are used herein interchangeably to describe an artificially assembled or isolated nucleic acid molecule which includes the polynucleotide of interest. In general a construct may include the polynucleotide or polynucleotides of interest, a marker gene which in some cases can also be a gene of interest and appropriate regulatory sequences. According to certain embodiments of the invention, the polynucleotide of interest encode siRNA molecule. It should be appreciated that the inclusion of regulatory sequences in a construct is optional, for example, such sequences may not be required in situations where the regulatory sequences of a host cell are to be used. The regulatory elements typically include a promoter sequence for directing transcription of the polynucleotide of interest in the cell in a constitutive or inducible manner. The term construct includes vectors but should not be seen as being limited thereto. According to certain embodiments, the term “vector,” is intended to refer to a nucleic acid molecule capable of transporting another nucleic acid to which it has been linked One type of vector is a “plasmid.” which refers to a circular double stranded DNA loop into which additional DNA segments may be ligated Another type of vector is a phage vector. Another type of vector is a viral vector, wherein additional DNA segments may be ligated into the viral genome (such as an adenoviral vector, a lentiviral vector, etc.). Certain vectors are capable of autonomous replication in a host cell into which they are introduced (e.g., bacterial vectors having a bacterial origin of replication and episomal mammalian vectors). Other vectors (e.g., non-episomal mammalian sectors) can be integrated into the genome of a host cell upon introduction into the host cell and thereby are replicated along with, the host genome.

The terms “complementary” or “complement thereof” are used herein to refer to the sequences of polynucleotides which is capable of forming Watson & Crick base pairing with another specified polynucleotide throughout the entirety of the complementary region. This term is applied to pairs of polynucleotides based solely upon their sequences and not any particular set of conditions under which the two polynucleotides would actually bind. The terms “substantially complementary” and “sufficiently complementary” are used herein interchangeably. An oligomeric compound need not be 100% complementary to its target nucleic acid to be specifically hybridizable. Moreover, an oligomeric compound may hybridize over one or more segments such that intervening or adjacent segments are not involved in the hybridization (e.g., a bulge, a loop structure or a hairpin, structure) A “non-complementary nucleobase” means a nucleobase of an antisense oligonucleotide that is unable to undergo precise base pairing with a nucleobase at a corresponding position in a target nucleic acid. In some embodiments there are non-complementary positions, also known as “mismatches”, between the oligomeric compound and the target nucleic acid, and such non-complementary positions may be tolerated between an oligomeric compound and the target nucleic acid provided that the oligomeric compound remains substantially complementary to the target nucleic acid.

The term “expression”, as used herein, refers to the production of a functional end-product e.g., an mRNA or a protein.

According to one aspect, the present invention provides a method of reducing the tumorigenicity of a tumor, the method comprises administering to, or expressing in cells of a subject affected with a tumor comprising cancer stem cells (CSCs) an effective amount of at least one voltage dependent anion channel 1 (VDAC1)-silencing oligonucleotide, thereby reducing the number and/or frequency of the CSCs in said tumor.

According to another aspect, the present invention provides a method for inhibiting cancer stem cells within a tumor, the method comprises administering to, or expressing in a tumor comprising cancer stem cells (CSCs) an effective amount of at least one voltage dependent anion channel 1 (VDAC1)-silencing oligonucleotide.

Without wishing to be bound by any specific theory or mechanism of action, inhibition of the CSCs within a tumor is the outcome of inhibition and/or reduction in the number of CSCs by inducing CSC differentiation leading to reduced heterogenicity in the cancer cell population on phenotypical level.

According to certain embodiments, the tumor is a non-solid tumor. According to some embodiments, the non-solid tumor is blood cancer.

According to other embodiments, the tumor is a solid tumor. According to certain embodiments, the solid tumor is selected from the group consisting of brain, breast, prostate, cervical, ovary, pancreas, head and neck, lymphoma, melanoma and colon cancer. Each possibility represents a separate embodiment of the present invention. According to certain embodiments, the brain tumor is glioma. According to certain exemplary embodiments, the glioma is glioblastoma multiforme (GBM).

According to yet additional aspect, the present invention provides a method of treating a subject with metastatic cancer or at risk of developing metastatic cancer, the method comprises administrating to, or expressing in cells of the subject an effective amount of at least one voltage dependent anion channel 1 (VDAC1)-silencing oligonucleotide.

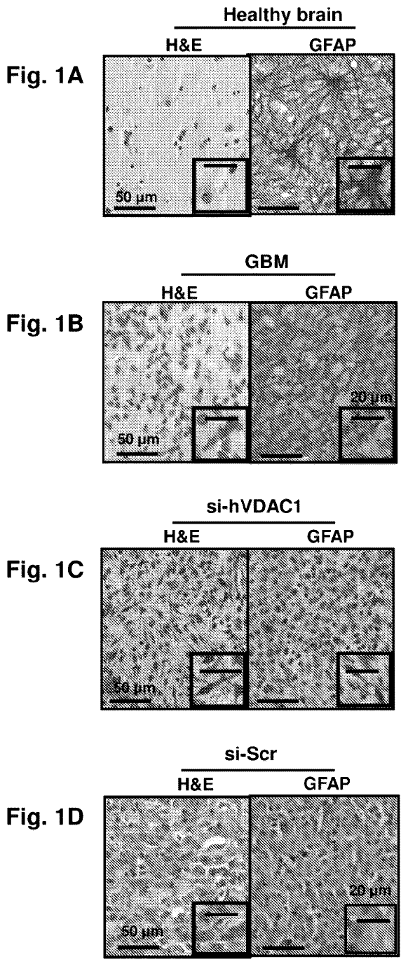

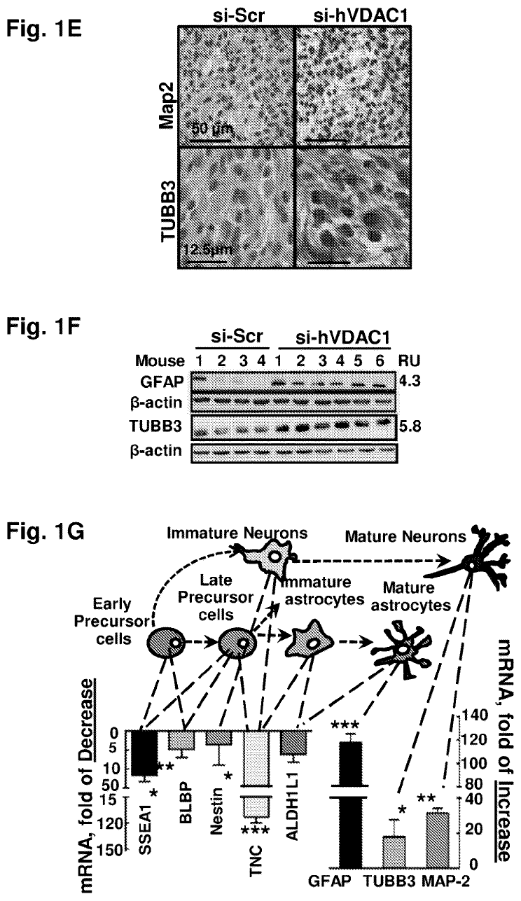

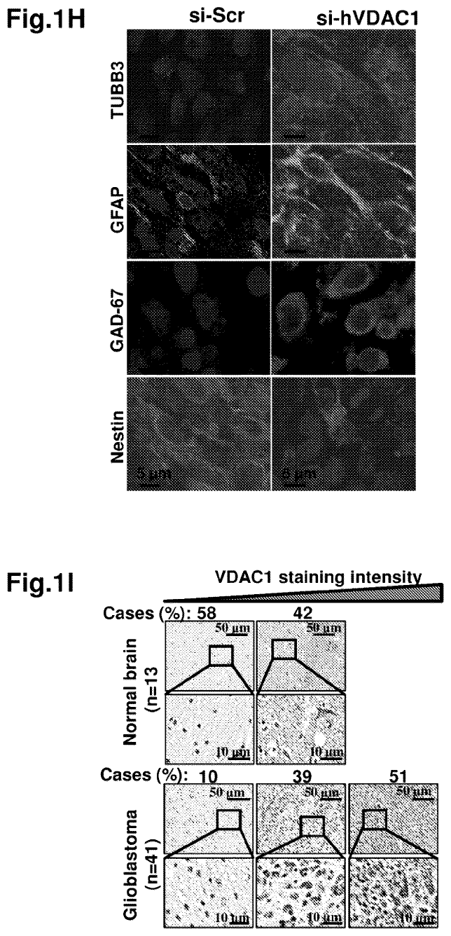

The central role of VDAC1 in cell energy and metabolism is reflected in its over-expression in many tumors, including glioma and in VDAC1 depletion impairing cancer cell energy and metabolic homeostasis (U.S. Pat. No. 8,093,369; Arif T, 2014, ibid; Shoshan-Barmatz et al., 2015, ibid. FIG. 1I).

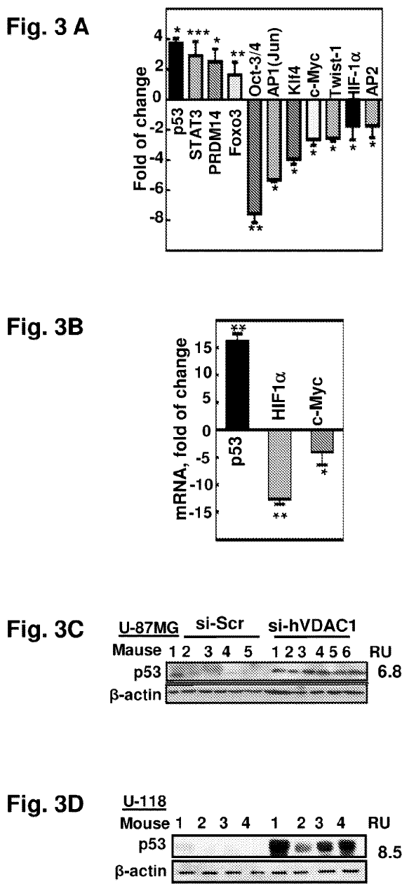

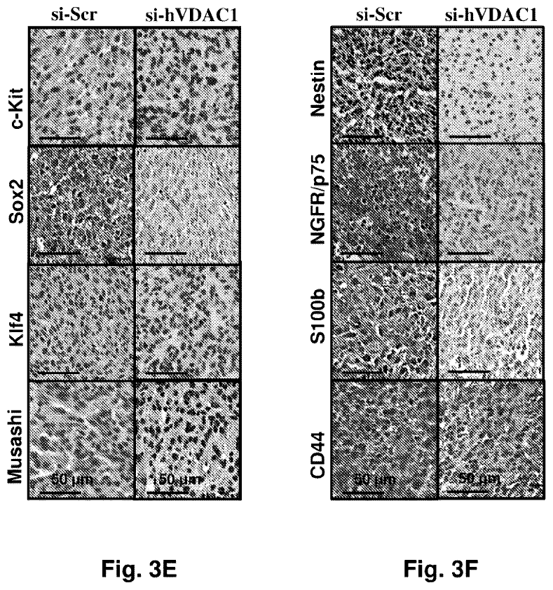

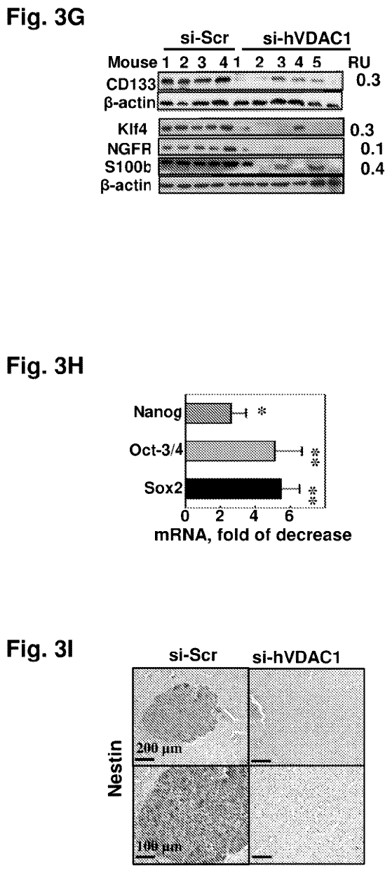

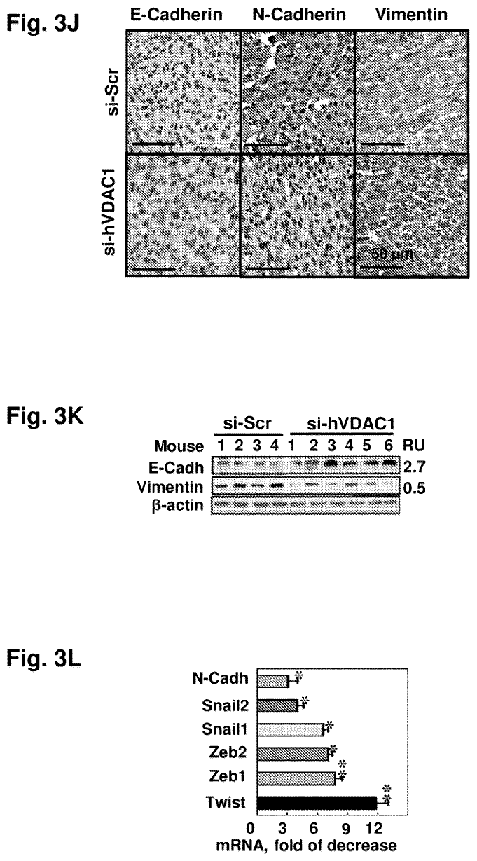

Recent studies have reinforced the hypothesis that human gliomas contain a neural stem cell lineage (Dirks P B., 2010. Molecular oncology 4, 420-430) capable of self-renewal and differentiation into multiple lineages (Chen J et al., 2012. Nature 488, 522.526). Unexpectedly, as exemplified herein (FIG. 3E-I), tumor treatment with siRNA targeted to hVDAC1 (si-hVDAC1) apparently eliminated GSCs, as reflected in the high decrease in expression of factors known to be involved in sternness and invasion (Shiras A et al., 2003. Neoplasia 5, 520-532). These included CD133, c-Kit. Sox2. Musashi, S100b. Klf4, Oct3/4, Nestin, CD44. Nanog and NGFR (FIG. 3E-G). Oct3/4 and Nanog are the earliest expressed set of genes known to control stemness and repress genes essential for development and/or differentiation (Sun Y et al., 2006. Critical reviews in eukaryotic gene expression 16, 211-231). CD133 is used as a pluripotent stem cell marker associated with an aggressive radio- and chemo-resistant sub-population of GBM cells (Yan X et al., 2011. PNAS LISA 108, 1591-1596). The decrease in GSC levels upon si-hVDAC1 treatment would thus make GBM more sensitive to irradiation and chemotherapy and addresses any concern about the tumor-forming potential of pluripotent stem cells. The decrease in GSC levels could be due to proliferation inhibition and/or differentiation, as reflected in morphological changes and increased expression of the neuronal differentiation markers GFAP, MAP-2, TUBB3 and GAD67 (FIGS. 1, 2, 3).

VDAC1 depletion leading to cell differentiation is further supported by bioinformatics promoter analysis of genes differentially expressed in si-hVDAC1-treated tumors (TTs), pointing to enrichment for binding sites of TFs involved in cell differentiation (FIG. 3A-D). FOXP1 was over-expressed in a cohort of GBM patients, with its silencing leading to tumor growth inhibition (Gomez G G et al., 2014. Cancer Research 74, 1429-1439). NRSF was also found to be enriched in GBM, with its knockdown strongly reducing tumor-initiating capacity in vivo (Binello E and Germano, I M., 2012. Neuro-oncology 14, 256-265). Finally, HNF4 and MAZ were reported to co-regulate neural stem cell differentiation via Rho-GDIγ (Wang J et al., 2013. Molecular Neurobiology 47, 228-240). As exemplified hereinbelow, these TFs were down-regulated in si-hVDAC1-TTs, thus leading to cell differentiation.

Hence, based on cell morphological changes, increased expression of mature neuronal and astrocytic markers and the decreased expression of GSCs markers (FIG. 3), it is proposed that upon VDAC1 depletion, GSCs in U-87MG cell-derived tumors are capable of in vivo neuronal differentiation toward mature neurons and astrocytes. This finding of the present invention is of high significance, as end-stage neurons cannot replicate, thereby preventing tumor re-growth and relapse.

According to certain embodiments, the VDAC1-silencing oligonucleotide comprises at least 15 contiguous nucleic acids identical to the gene or mRNA encoding human VDAC1 protein or to a complementary polynucleotide thereof, wherein the human VDAC1 protein comprises the amino acid sequence set forth in SEQ ID NO:1.

According to certain embodiments the hVDCA1 protein is encoded by the nucleic acid sequence set forth in SEQ ID NO:2.

The terms “VDAC1-silencing oligonucleotide”, “VDAC1-silencing oligonucleotide molecule”, “VDAC1-silencing molecule” or “oligonucleic that inhibits or reduces VDAC1 expression” as used herein, denote an oligonucleic acid capable of specifically reducing the level or expression of the gene product, i.e. the level of VDAC1 RNA, below the level that is observed in the absence of the oligonucleic acid. In some embodiments gene expression is down-regulated by at least 25%, preferably at least 50%, at least 70%, 80% or at least 90%.

The siRNA designated si-hVDAC1 in the figures and specification of the present invention refers to siRNA comprising a first oligonucleotide having the nucleic acid sequence set forth in SEQ ID NO:14 (the “sense” oligonucleotide) and a second oligonucleotide having the nucleic acid sequence set forth in SEQ ID NO: 15 (the “antisense” oligonucleotide).

It is to be explicitly understood that while silencing the expression of VDAC1 as described herein uses RNAi as silencing oligonucleotide molecules, other methods for silencing the expression of VDAC1 including inhibiting transcription from the VDAC1 gene to VDCA1 RNA; by inhibition of the maturation process from hnRNA to mRNA; by promotion of mRNA degradation in the cytoplasm by enzymes (by forming RNA duplexes or triplexes, and by catalytic cleavage of nucleic acid based enzymes (DNAzymes and RNAzymes).

According to certain exemplary embodiments, the VDAC1-silencing oligonucleotide is RNAi molecule. According to additional exemplary embodiments, the RNAi molecule is selected from the group consisting of siRNA and shRNA. Each possibility represents a separate embodiment of the present invention.

As used herein the term “siRNA” (small interfering RNA) refers to a nucleic acid that forms a double stranded RNA, wherein the double stranded RNA has the ability to reduce or inhibit expression of a particular gene or target gene when the siRNA is present or expressed in the same cell as the gene or target gene. The double stranded RNA siRNA can be formed by the complementary strands. The complementary portions of the siRNA that hybridize to form the double stranded molecule typically have substantial or complete identity. In one embodiment, a siRNA refers to a nucleic acid that has substantial or complete identity to sequence of a target gene and forms a double stranded RNA. The sequence of the siRNA can correspond to the full length target gene, or to a subsequence thereof. Typically, the siRNA is at least about 15-50 nucleotides in length (e.g., each complementary sequence of the double stranded siRNA is about 15-50 nucleotides in length, and the double stranded siRNA is about 15-50 base pairs in length, preferably about 19-30 base nucleotides, preferably about 19-25 nucleotides in length, e.g., 19, 20, 21, 22, 23, 24, 25, 26, 27, 28, 29, or 30 nucleotides in length).

As used herein “shRNA” or “small hairpin RNA” (also called stem loop) is a type of siRNA. In one embodiment, these shRNAs are composed of a short, e.g. about 19 to about 25 nucleotides, antisense strand, followed by a nucleotide loop or about 5 to about 9 nucleotides, and the analogous sense strand. Alternatively, the sense strand can precede the nucleotide loop structure and the antisense strand can follow.

As illustrated it Table 1 hereinbelow, certain exemplary silencing oligonucleotide have been designed, targeted to specific areas of the VDAC1 gene (hybridizable with specific areas of the VDAC1 transcript) and substantially comprise a nucleic acid sequence as set forth in any one of SEQ ID NOs:3-10.

TABLE 1
Exemplary VDAC1-silencing oligonucleotide (sense strand)

| | SEQ ID | | SEQ ID | |
|---|---|---|---|---|
| Sense Sequence | NO: | Target VDAC1 Sequence | NO: | Location |
| ACACUAGGCACCGAGAUUA | 3 | ACACTAGGCACCGAGATTA | 16 | 238-256 |
| GGGCUAUGGAUUUGGCUUA | 4 | GGGCTATGGATTTGGCTTA | 17 | 159-177 |
| GCUUGGUCUAGGACUGGAA | 5 | GCTTGGTCTAGGACTGGAA | 18 | 921-939 |
| AAGCUGACCUUCGAUUCAU | 6 | AAGCTGACCTTCGATTCAT | 19 | 531-549 |
| GAAUGACGGGACAGAGUUU | 7 | GAATGACGGGACAGAGTTT | 20 | 797-815 |
| UCGGAAUAGCAGCCAAGUA | 8 | TCGGAATAGCAGCCAAGTA | 21 | 901-919 |
| CUCUUCUGGAUGGCAAGAA | 9 | CTCTTCTGGATGGCAAGAA | 22 | 1027-1045 |
| GAAUAGCAGCCAAGUAUCAG | 10 | GAATAGCAGCCAAGTATCAG | 23 | 487-505 |

According to additional embodiments, at least one of the siRNA nucleic acids is chemically modified. Typically, the modification is 2′-O-methyl modification of a guanine or uracil. According to certain embodiments, the first and the second polynucleotide of the RNAi comprise several chemically modified guanine and/or uracil nucleotides. According to certain exemplary embodiments, the modified siRNA molecule comprises a first oligonucleotide having the nucleic acid sequence set forth in SEQ ID NO:14 and a second oligonucleotide having the nucleic acid sequence set forth in SEQ TD NO 15.

According to certain embodiments, reducing the tumorigenicity of the tumor comprises attenuating the invasive potential of said tumor, particularly of the tumor cancer stem cells.

The epithelial-mesenchymal transition (EMT) plays a critical role in embryonic development. EMT is also involved in cancer progression and metastasis and it is probable that a common molecular mechanism is shared by these processes. Cancer cells undergoing EMT can acquire invasive properties and enter the surrounding stroma, resulting in the creation of a favorable microenvironment for cancer progression and metastasis. Furthermore, the acquisition of EMT features has been associated with chemoresistance which could give rise to recurrence and metastasis after standard chemotherapeutic treatment. Thus, EMT could be closely involved in carcinogenesis, invasion, metastasis, recurrence, and chemoresistance (Iwatsuki, M et al., 2010. Cancer science 101, 293-299).

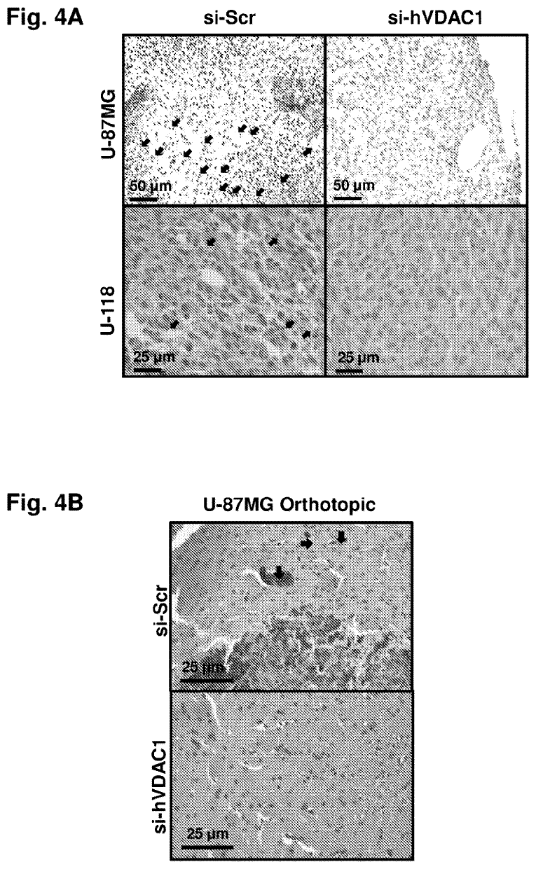

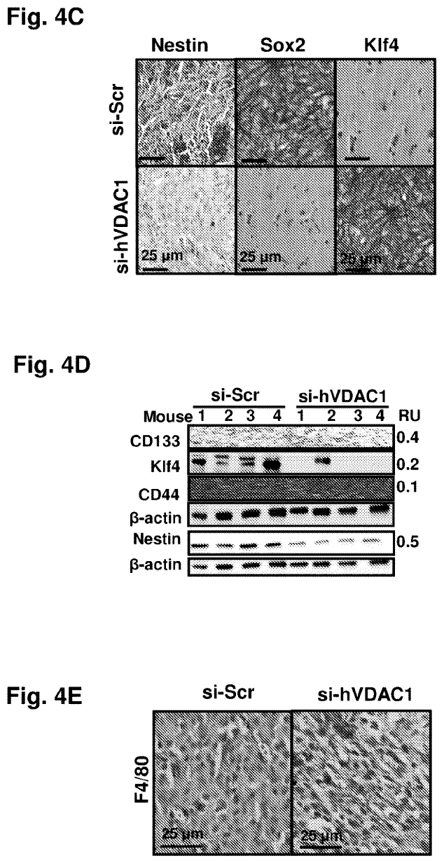

Treating tumors with siRNA targeted to hVDAC1 changed the expression of proteins involved in EMT and of extracellular matrix (ECM) components (Table 3). Tumor treatment with si-hVDAC also decreased the levels of tumor-associated macrophage (TAM) markers (F4/80, CD68), suggesting the tumors possess less pro-tumorigenic activity (inflammation, ROS, cytokine, growth factors). Among cells of immune system, macrophages are particularly abundant and are present at all stages of tumor progression. Macrophages generally have a pro-tumoral role. In the primary tumor, macrophages take substantial task in angiogenesis stimulation and enhance tumor cell motility and invasion (Noy R and Pollard J W. 2014. Immunity 41(1), 49-61). Without wishing to be bound by any specific theory or mechanism of action, the reduction in the pro-tumorigenic activity also decreased the angiogenesis potential (Glass R and Synowitz, M., 2014. Glia 62, 514-525). Indeed, VDAC1 depletion resulted in a strong decrease in angiogenesis, both in microvessel density and VEGF levels (FIG. 4E-H). VEGF is highly over-expressed in GBM (Rahmathulla G et al., 2013. OncoTargets and therapy 6, 371-389). Anti-angiogenesis therapy, like VEGFR inhibitors/antibodies, has been a focal area in cancer drug discovery for over a decade, including as GBM therapy, albeit with limited success or organ-specific toxicity. The present invention now discloses that RNAi molecules targeted to silence VDAC1 expression can be used as efficient anti-angiogenesis drugs.

Changes in cancer cell metabolism contribute to the oncogenic process. The metabolic changes support the three basic needs of dividing cancer cells; rapid ATP generation to maintain energy status; increased biosynthesis of macromolecules; and tightened maintenance of appropriate cellular redox status. VDAC1 is a key protein in regulating cancer cell energy and has been shown to be over-expressed in cancer cells including glioblastoma. VDAC1 is essential to mitochondrial ATP production and is the main transporter of ATP and other metabolites and ions through the outer mitochondrial membrane. Moreover, VDAC1 mediates cholesterol transport, with cancer cells exhibiting much higher cholesterol levels than do healthy cells. It has been previously described that silencing the expression of VDAC1 disrupts energy production and cell growth. The present invention now shows that silencing VDAC1 expression resulted in down regulation of glucose transporter, glycolytic enzymes, and modified expression profile transcription factors regulating metabolism. In particular, the present invention shows that silencing VDAC1 resulted in dramatic decrease in the expression level of the glucose transported Glut-1, facilitating glucose uptake by the cells and of the glycolytic enzymes hexokinase (HK-1), glyceraldehyde dehydrogenase (GAPDH) and lactate dehydrogenase (LDH), all known to be up-regulated in cancer cells.

In summary, the present invention now shows that unexpectedly, VDAC1 depletion reversed the reprogrammed energy and metabolism characteristic to cancer cells, reinforced cell differentiation and reduced stemness, invasion, TAMs abundance and angiogenesis.

### Silencing Molecules

The silencing oligonucleotide molecules designed according to the teachings of the present invention can be generated according to any nucleic acid synthesis method known in the art, including both enzymatic syntheses and solid-phase syntheses. Any other means for such synthesis may also be employed; the actual synthesis of the nucleic acid agents is well within the capabilities of one skilled in the art and can be accomplished via established methodologies as detailed in, for example; Sambrook. J. and Russell, D. W. (2001). “Molecular Cloning: A Laboratory Manual”; Ausubel, R. M. et al., eds. (1994, 1989). “Current Protocols in Molecular Biology,” Volumes I-III, John Wiley & Sons, Baltimore, Maryland; Perbal. B. (1988), “A Practical Guide to Molecular Cloning,” John Wiley & Sons, New York; and Gait, M. J., ed. (1984). “Oligonucleotide Synthesis”; utilizing solid-phase chemistry, e.g. cyanoethyl phosphoramidite followed by deprotection, desalting, and purification by, for example, an automated trityl-on method or HPLC.

It will be appreciated that nucleic acid agents of the present invention can be also generated using an expression vector as is further described hereinbelow.

According to certain embodiments, the silencing oligonucleotide molecules of the present invention are modified. Nucleic acid agents can be modified using various methods known in the art.

For example, the silencing oligonucleotide molecules of the present invention may comprise heterocylic nucleosides consisting of purines and the pyrimidines bases, bonded in a 3′-to-5′ phosphodiester linkage.

Preferably used nucleic acid agents are those modified either in backbone, internucleoside linkages, or bases, as is broadly described hereinbelow.

Specific examples of silencing oligonucleotide molecules useful according to this aspect of the present invention include oligonucleotides or polynucleotides containing modified backbones or non-natural internucleoside linkages. Oligonucleotides or polynucleotides having modified backbones include those that retain a phosphorus atom in the backbone, as disclosed in U.S. Pat. Nos. 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050.

Preferred modified oligonucleotide backbones include, for example: phosphorothioates; chiral phosphorothioates; phosphorodithioates; phosphotriesters; aminoalkyl phosphotriesters; methyl and other alkyl phosphonates, including 3′-alkylene phosphonates and chiral phosphonates; phosphinates; phosphoramidates, including 3′-amino phosphoramidate and aminoalkylphosphoramidates; thionophosphoramidates; thionoalkylphosphonates; thionoalkylphosphotriesters; and boranophosphates having normal 3′-5′ linkages, 2‘-’ linked analogues of these, and those having inverted polarity wherein the adjacent pairs of nucleoside units are linked 3′-5′ to 5′-3′ or 2′-5′ to 5′-2′. Various salts, mixed salts, and free acid forms of the above modifications can also be used.

Alternatively, modified oligonucleotide backbones that do not include a phosphorus atom therein have backbones that are formed by short-chain alkyl or cycloalkyl internucleoside linkages, mixed heteroatom and alkyl or cycloalkyl internucleoside linkages, or one or more short-chain heteroatomic or heterocyclic internucleoside linkages. These include those having morpholino linkages (formed in part from the sugar portion of a nucleoside); siloxane backbones; sulfide, sulfoxide, and sulfone backbones; formacetyl and thioformacetyl backbones; methylene formacetyl and thioformacetyl backbones; alkene-containing backbones; sulfamate backbones; methyleneimino and methylenehydrazino backbones; sulfonate and sulfonamide backbones; amide backbones; and others having mixed N, O, S and CH2 component parts, as disclosed in U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439.

Other silencing oligonucleotide molecules which may be used according to the present invention are those modified in both sugar and the internucleoside linkage, i.e., the backbone of the nucleotide units is replaced with novel groups. The base units are maintained for complementation with the appropriate polynucleotide target. An example of such an oligonucleotide mimetic includes a peptide nucleic acid (PNA). A PNA oligonucleotide refers to an oligonucleotide where the sugar-backbone is replaced with an amide-containing backbone, in particular an aminoethylglycine backbone. The bases are retained and are bound directly or indirectly to aza-nitrogen atoms of the amide portion of the backbone. United States patents that teach the preparation of PNA compounds include, but are not limited to, U.S. Pat. Nos. 5,539,082; 5,714,331; and 5,719,262; each of which is herein incorporated by reference. Other backbone modifications which may be used in the present invention are disclosed in U.S. Pat. No. 6,303,374.

Silencing oligonucleotide molecules of the present invention may also include base modifications or substitutions. As used herein, “unmodified” or “natural” bases include the purine bases adenine (A) and guanine (G) and the pyrimidine bases thy mine (T), cytosine (C), and uracil (U). “Modified” bases include but are not limited to other synthetic and natural bases, such as: 2′-O-methyl-modified nucleotides, particularly uracil and guanine; 5-methylcytosine (5-me-C); 5-hydroxymethyl cytosine; xanthine; hypoxanthine; 2-aminoadenine; 6-methyl and other alkyl derivatives of adenine and guanine; 2-propyl and other alkyl derivatives of adenine and guanine; 2-thiouracil, 2-thiothymine, and 2-thiocytosine; 5-halouracil and cytosine; 5-propynyl uracil and cytosine; 6-azo uracil, cytosine, and thymine; 5-uracil (pseudouracil); 4-thiouracil; 8-halo, 8-amino, 8-thiol, 8-thioalkyl, 8-hydroxyl, and other 8-substituted adenines and guanines; 5-halo, particularly 5-bromo, 5-trifluoromethyl, and other 5-substituted uracils and cytosines; 7-methylguanine and 7-methyladenine; 8-azaguanine and 8-azaadenine; 7-deazaguanine and 7-deazaadenine; and 3-deazaguanine and 3-deazaadenine. Additional modified bases include those disclosed in: U.S. Pat. No. 3,687,808; Kroschwitz, J I., ed. (1990), “The Concise Encyclopedia Of Polymer Science And Engineering,” pages 858-859, John Wiley & Sons; Englisch et al. (1991), “Angewandte Chemie,” International Edition, 30, 613; and Sanghvi. Y. S., “Antisense Research and Applications,” Chapter 15, pages 289-302, S. T. Crooke and B. Lebleu, eds., CRC Press, 1993. Such modified bases are particularly useful for increasing the binding affinity of the oligomeric compounds of the invention. These include 5-substituted pyrimidines, 6-azapyrimidines, and N-2, N-6, and O-6-substituted purines, including 2-aminopropyladenine, 5-propynyluracil, and 5-propynylcytosine. 5-methylcytosine substitutions have been shown to increase nucleic acid duplex stability by 0.6-1.2° C. (Sanghvi, Y. S. et al. (1993). “Antisense Research and Applications,” pages 276-278, CRC Press, Boca Raton), and are presently preferred base substitutions, even more particularly when combined with 2′-O-methoxyethyl sugar modifications.

The silencing oligonucleotide molecules of the present invention are of at least 10, at least 15, or at least 17 bases specifically hybridizable with VDCA1 RNA. According to certain exemplary embodiments, the siRNAs of the present invention are of 19 bases.

It should be appreciated that the present invention also envisages agents other than nucleic acid agents that are capable of down-regulating VDCA1 RNA such as knock-out agents.

Synthesis of RNAi molecules suitable for use with the present invention can be effected as follows. First, the VDAC1 nucleic acid sequence target is scanned downstream for AA dinucleotide sequences. Occurrence of each AA and the 3′ adjacent 19 nucleotides is recorded as potential siRNA target sites.

Second, potential target sites are compared to an appropriate genomic database (e.g., human, mouse, rat etc.) using any sequence alignment software, such as the BLAST software available from the NCBI server (www.ncbi.nlm.nih.gov/BLAST/). Putative target sites that exhibit significant homology to other coding sequences are filtered out.

Qualifying target sequences are selected as template for siRNA synthesis. Preferred sequences are those including low G/C content as these have proven to be more effective in mediating gene silencing as compared to those with G/C content higher than 55%. Several target sites are preferably selected along the length of the target gene for evaluation. For better evaluation of the selected siRNAs, a negative control is preferably used in conjunction. Negative control siRNA preferably include the same nucleotide composition as the siRNAs but lack significant homology to the genome. Thus, a scrambled nucleotide sequence of the siRNA is preferably used, provided it does not display any significant homology to any other gene.

Examples of siRNAs which are capable of down-regulating VDAC1 that may be used according to this aspect of the present invention are those set forth by SEQ ID NOs:3-10 and sequences essentially complementary thereto.

Another agent capable of downregulating the expression of a VDAC1 RNA is a DNAzyme molecule capable of specifically cleaving its encoding polynucleotide. DNAzymes are single-stranded nucleic acid agents which are capable of cleaving both single and double stranded target sequences (Breaker R R and Joyce, G. 1995. Chemistry and Biology 2, 655; Santoro, S. W. & Joyce, G. F. Proc. Natl, Acad. Sci. USA 1997; 94:4262). A general model (the “10-23” model) for the DNAzyme has been proposed. “10-23” DNAzymes have a catalytic domain of 15 deoxynbonucleotides, flanked by two substrate-recognition domains of seven to nine deoxyribonucleotides each. This type of DNAzyme can effectively cleave its substrate RNA at purine:pyrimidine junctions (Santoro. S. W. & Joyce, G. F. Proc. Natl, Acad. Sci. USA 199; for rev of DNAzymes see Khachigian, L M (2002. Curr Opin Mol Ther 4, 119˜21).

Examples of construction and amplification of synthetic, engineered DNAzymes recognizing single and double-stranded target cleavage sites have been disclosed in U.S. Pat. No. 6,326,174 to Joyce et al. DNAzymes of similar design directed against the human Urokinase receptor were recently observed to inhibit Urokinase receptor expression, and successfully inhibit colon cancer cell metastasis in vivo (Itoh et al., 20002, Abstract 409, Ann Meeting Am Soc Gen Ther www.asgt.org). In another application, DNAzymes complementary to bcr-ab1 oncogenes were successful in inhibiting the oncogenes expression in leukemia cells, and lessening relapse rates in autologous bone marrow transplant in cases of Chronic Myelogenous Leukemia (CML) and Acute Lymphocytic Leukemia (ALL).

Another agent capable of downregulating VDAC1 RNA is a ribozyme molecule capable of specifically cleaving its encoding polynucleotide. Ribozymes are being increasingly used for the sequence-specific inhibition of gene expression by the cleavage of mRNAs encoding proteins of interest (Welch et al., 1998. Curr Opin Biotechnol. 9:486˜96). The possibility of designing ribozymes to cleave any specific target RNA has rendered them valuable tools in both basic research and therapeutic applications. In the therapeutics area, ribozymes have been exploited to target viral RNAs in infectious diseases, dominant oncogenes in cancers and specific somatic mutations in genetic disorders (Welch et al., 1998. Clin Diagn Virol. 10:163-71). Most notably, several ribozyme gene therapy protocols for HIV patients are already in Phase I trials. More recently, ribozymes have been used for transgenic animal research, gene target validation and pathway elucidation. Several ribozymes are in various stages of clinical trials. ANGIOZYME was the first chemically synthesized ribozyme to be studied in human clinical trials. ANGIOZYME specifically inhibits formation of the VEGF-r (Vascular Endothelial Growth Factor receptor), a key component in the angiogenesis pathway. Ribozyme Pharmaceuticals, Inc., as well as other firms have demonstrated the importance of anti-angiogenesis therapeutics in animal models. HEPTAZYME, a ribozyme designed to selectively destroy Hepatitis C Virus (HCV) RNA, was found effective in decreasing Hepatitis C viral RNA in cell culture assays (Ribozyme Pharmaceuticals, Incorporated—http://www.rpi.com/index.html).

An additional method of downregulating VDAC1 RNA is via triplex forming oligonuclotides (TFOs). In the last decade, studies have shown that TFOs can be designed which can recognize and bind to poly purine/polypirimidine regions in double-stranded helical DNA in a sequence-specific manner. Thus the DNA sequence encoding the VDCA1 RNA of the present invention can be targeted thereby down-regulating the RNA molecule.

The recognition rules governing TFOs are outlined by Maher III, L. J., et al., Science (1989) 245:725-730; Moser, H. E., et al., Science (1987)238:645-630; Beal, P. A., et al., Science (1991) 251:1360-1363; Cooney, M., et al., Science (1988)241:456-459; and Hogan, M. E., et al., EP Publication 375408. Modification of the oligonuclotides, such as the introduction of intercalators and backbone substitutions, and optimization of binding conditions (pH and cation concentration) have aided in overcoming inherent obstacles to TFO activity such as charge repulsion and instability, and it was recently shown that synthetic oligonucleotides can be targeted to specific sequences (for a recent review see Seidman and Glazer (2003) J Clin Invest; 112:487-94).

In general, the triplex-forming oligonucleotide has the sequence correspondence:

| | |
|---|---|
| oligo | 3′--A G G T |
| duplex | 5′--A G C T |
| duplex | 3′--T C G A |

However, it has been shown that the A-AT and G-GC triplets have the greatest triple helical stability (Reither and Jeltsch (2002), BMC Biochem., September 12, Epub). The same authors have demonstrated that TFOs designed according to the A-AT and G-GC rule do not form non-specific triplexes, indicating that the triplex formation is indeed sequence specific.

Thus for any given sequence in the regulatory region a triplex forming sequence may be devised. Triplex-forming oligonucleotides preferably are at least 15, more preferably 25, still more preferably 30 or more nucleotides in length, up to 50 or 100 bp.

Transfection of cells (for example, via cationic liposomes) with TFOs, and subsequent formation of the triple helical structure with the target DNA, induces steric and functional changes, blocking transcription initiation and elongation, allowing the introduction of desired sequence changes in the endogenous DNA and results in the specific downregulation of gene expression. Examples of such suppression of gene expression in cells treated with TFOs include knockout of episomal supFG1 and endogenous HPRT genes in mammalian cells (Vasquez et al., Nucl Acids Res. (1999) 27:1176-81, and Puri, et al., J Biol Chem, (2001) 276:28991-98), and the sequence- and target-specific downregulation of expression of the Ets2 transcription factor, important in prostate cancer etiology (Carbone, et al., Nucl Acid Res. (2003) 31:833-43), and the pro-inflammatory ICAM-1 gene (Besch et al., J Biol Chem, (2002) 277:32473-79). In addition, Vuyisich and Beal have recently shown that sequence specific TFOs can bind to dsRNA, inhibiting activity of dsRNA-dependent enzymes such as RNA-dependent kinases (Vuyisich and Beal, Nuc. Acids Res 2000. 28:2369-74).

Additionally, TFOs designed according to the abovementioned principles can induce directed mutagenesis capable of effecting DNA repair, thus providing both downregulation and upregulation of expression of endogenous genes (Seidman and Glazer, J Clin Invest 2003. 112, 487-94). Detailed description of the design, synthesis and administration of effective TFOs can be found in U.S. Patent Application Nos. 2003 017068 and 2003 0096980 to Froehler et al., and 2002 0128218 and 2002 0123476 to Emanuele et al., and U.S. Pat. No. 5,721,138 to Lawn.

It will be appreciated that silencing oligonucleotide molecules capable of hybridizing VDCA1 mRNA may down-regulate an activity thereof by preventing VDCA1 mRNA binding to another downstream agent.

### Silencing Molecule Delivery and Expression

Naked inhibitory nucleic acid molecules, or analogs thereof, are capable of entering mammalian cells and inhibiting expression of a gene of interest. Nonetheless, it may be desirable to utilize a formulation that aids in the delivery of oligonucleotides or other nucleobase oligomers to cells (see, e.g., U.S. Pat. Nos. 5,656,611, 5,753,613, 5,785,992, 6,120,798, 6,221,959, 6,346,613, and 6,353,055).

According to certain exemplary embodiments, the silencing oligonucleotide molecules of the present invention, specifically siRNAs, are shielded and/or encapsulated in Poly(D,L-lactide-co-glycolide) (PLGA). Without wishing to be bound by any specific theory or mechanism of action, PGLA shielding or encapsulation enables the siRNA molecules to cross the blood brain barrier (BBB). As mentioned hereinabove, the nucleic acid agents of the present invention (e.g., an siRNA molecule such as those set forth by SEQ ID NO:3-15) can be expressed in cells.

It will be appreciated that the VDAC1 silencing oligonecleotides of the present invention may be expressed directly in the subject (i.e. in vivo gene therapy) or may be expressed ex vivo in a cell system (autologous or non-autologous) and then administered to the subject.

To express such an agent (i.e., to produce an RNA molecule) in mammalian cells, a nucleic acid sequence encoding the agents of the present invention is preferably ligated into a nucleic acid construct suitable for mammalian cell expression. Such a nucleic acid construct includes a promoter sequence for directing transcription of the polynucleotide sequence in the cell in a constitutive or inducible manner.

Constitutive promoters suitable for use with the present invention are promoter sequences which are active under most environmental conditions and most types of cells such as the cytomegalovirus (CMV) and Rous sarcoma virus (RSV). Inducible promoters suitable for use with the present invention include for example the tetracycline-inducible promoter (Zabala M. et al., Cancer Res. 2004, 64(8): 2799-804).

The nucleic acid construct (also referred to herein as an “expression vector”) of the present invention includes additional sequences which render this vector suitable for replication and integration in prokaryotes, eukaryotes, or preferably both (e.g., shuttle vectors). In addition, typical cloning vectors may also contain a transcription and translation initiation sequence, transcription and translation terminator and a polyadenylation signal.

Eukaryotic promoters typically contain two types of recognition sequences, the TATA box and upstream promoter elements. The TATA box, located 25-30 base pairs upstream of the transcription initiation site, is thought to be involved in directing RNA polymerase to begin RNA synthesis. The other upstream promoter elements determine the rate at which transcription is initiated.

Preferably, the promoter utilized by the nucleic acid construct of the present invention is active in the specific cell population transformed. Examples of cell type-specific and/or tissue-specific promoters include promoters such as albumin that is liver specific (Pinkert et al., 1987. Genes Dev. 1, 268-277 0, lymphoid specific promoters 9Calame et al., 1988. Adv. Immunol. 43, 235-275 0; in particular promoters of T-cell receptors 9Winoto et al., 1989. EMBO J. 8, 729-733 0 and immunoglobulins; (Banerji et al., 1983. Cell 33, 729-740), neuron-specific promoters such as the neurofilament promoter (Byrne et al. 1989. Proc. Natl. Acad. Sci. USA 86, 5473-5477), pancreas-specific promoters (Edlunch et al, 1985. Science 230, 912-916) or mammary gland-specific promoters such as the milk whey promoter (U.S. Pat. No. 4,873,316 and European Application Publication No. 264.166).

Enhancer elements can stimulate transcription up to 1,000 fold from linked homologous or heterologous promoters. Enhancers are active when placed downstream or upstream from the transcription initiation site. Many enhancer elements derived from viruses have a broad host range and are active in a variety of tissues. For example, the SV40 early gene enhancer is suitable for many cell types. Other enhancer/promoter combinations that are suitable for the present invention include those derived from polyoma virus, human or murine cytomegalovirus (CMV), the long term repeat from various retroviruses such as murine leukemia virus, murine or Rous sarcoma virus and HIV. See, Enhancers and Eukaryotic Expression, Cold Spring Harbor Press, Cold Spring Harbor, N.Y. 1983.

In the construction of the expression vector, the promoter is preferably positioned approximately the same distance from the heterologous transcription start site as it is from the transcription start site in its natural setting. As is known in the art, however, some variation in this distance can be accommodated without loss of promoter function.

Polyadenylation sequences can also be added to the expression vector in order to increase RNA stability (Soreq et al., 1974. J. Mol Biol. 88: 233-45).

Two distinct sequence elements are required for accurate and efficient polyadenylation: GU or U rich sequences located downstream from the polyadenylation site and a highly conserved sequence of six nucleotides, AAUAAA, located 11-30 nucleotides upstream. Termination and polyadenylation signals that are suitable for the present invention include those derived from SV40.

In addition to the elements already described, the expression vector of the present invention may typically contain other specialized elements intended to increase the level of expression of cloned nucleic acids or to facilitate the identification of cells that carry the recombinant DNA. For example, a number of animal viruses contain DNA sequences that promote the extra chromosomal replication of the viral genome in permissive cell types. Plasmids bearing these viral replicons are replicated episomally as long as the appropriate factors are provided by genes either carried on the plasmid or with the genome of the host cell.

The vector may or may not include a eukaryotic replicon. If a eukaryotic replicon is present, then the vector is amplifiable in eukaryotic cells using the appropriate selectable marker. If the vector does not comprise a eukaryotic replicon, no episomal amplification is possible. Instead, the recombinant DNA integrates into the genome of the engineered cell, where the promoter directs expression of the desired nucleic acid.

Examples for mammalian expression vectors include, but are not limited to, pcDNA3, pcDNA3.1(+/−), pGL3, pZeoSV2(+/−), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

Expression vectors containing regulatory elements from eukaryotic viruses such as retroviruses can be also used. SV40 vectors include pSVT7 and pMT2. Vectors derived from bovine papilloma virus include pBV-IMTHA, and vectors derived from Epstein Bar virus include pHEBO, and p2O5. Other exemplary vectors include pMSG, pAV009/A+, pMTO10/A+, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

As described above, viruses are very specialized infectious agents that have evolved, in many cases, to elude host defense mechanisms. Typically, viruses infect and propagate in specific cell types. The targeting specificity of viral vectors utilizes its natural specificity to specifically target predetermined cell types and thereby introduce a recombinant gene into the infected cell. Thus, the type of vector used by the present invention will depend on the cell type transformed. The ability to select suitable vectors according to the cell type transformed is well within the capabilities of the ordinary skilled artisan and as such no general description of selection consideration is provided herein. For example, bone marrow cells can be targeted using the human T cell leukemia virus type I (HTLV-I) and kidney cells may be targeted using the heterologous promoter present in the baculovirus Autographa californica nucleopolyhedrovirus (AcMNPV) as described in Liang C Y et al., 2004 (Arch Virol. 149: 51-60).

Recombinant viral vectors are useful for in vivo expression of the VDCA1 silencing molecules of the present invention since they offer advantages such as lateral infection and targeting specificity. Lateral infection is inherent in the life cycle of, for example, retrovirus and is the process by which a single infected cell produces many progeny virions that bud off and infect neighboring cells. The result is that a large area becomes rapidly infected, most of which was not initially infected by the original viral particles. This is in contrast to vertical-type of infection in which the infectious agent spreads only through daughter progeny. Viral vectors can also be produced that are unable to spread laterally. This characteristic can be useful if the desired purpose is to introduce a specified gene into only a localized number of targeted cells.

Various methods can be used to introduce the expression vector of the present invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995). Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. (Biotechniques 4 (6): 504-512, 1986) and include, for example, stable or transient transfection, lipofection, electroporation and infection with recombinant viral vectors. In addition, see U.S. Pat. Nos. 5,464,764 and 5,487,992 for positive-negative selection methods.

Introduction of nucleic acids by viral infection offers several advantages over other methods such as lipofection and electroporation, since higher transfection efficiency can be obtained due to the infectious nature of viruses.

Currently preferred in vivo nucleic acid transfer techniques include transfection with viral or non-viral constructs, such as adenovirus, lentivirus, Herpes simplex I virus, or adeno-associated virus (AAV) and lipid-based systems. Useful lipids for lipid-mediated transfer of the gene are, for example, DOTMA, DOPE, and DC-Chol (Tonkinson et al., 1996. Cancer Investigation, 14(1), 54-65). The most preferred constructs for use in gene therapy are viruses, most preferably adenoviruses, AAV, lentiviruses, or retroviruses. A viral construct such as a retroviral construct includes at least one transcriptional promoter/enhancer or locus-defining element(s), or other elements that control gene expression by other means such as alternate splicing, nuclear RNA export, or post-translational modification of messenger. Such vector constructs also include a packaging signal, long terminal repeats (LTRs) or portions thereof, and positive and negative strand primer binding sites appropriate to the virus used, unless it is already present in the viral construct. Optionally, the construct may also include a signal that directs polyadenylation, as well as one or more restriction sites. By way of example, such constructs will typically include a 5′ LTR, a tRNA binding site, a packaging signal, an origin of second-strand DNA synthesis, and a 3′ LTR or a portion thereof. Other vectors can be used that are non-viral, such as cationic lipids, polylysine, and dendrimers.

Other than containing the necessary elements for the transcription of the inserted coding sequence, the expression construct of the present invention can also include sequences engineered to enhance stability, production, purification, yield or toxicity of the expressed RNA.

The agents of the present invention can be administered to a subject per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism. According to the present invention, “an active ingredient” refers to VDAC1-silencing oligo nucleotide molecule.

The terms “physiologically acceptable carrier” and “pharmaceutically acceptable carrier” which may be interchangeably used herein refer to a carrier or a diluent that does not cause significant irritation to an organism and does not abrogate the biological activity and properties of the administered compound. An adjuvant is included under these phrases.

The term “excipient” as is used herein refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences.” Mack Publishing Co., Easton, PA, latest edition, which is incorporated herein by reference.

Suitable routes of administration may, for example, include parenteral, transmucosal, especially transnasal, intestinal, rectal, or oral delivery, including intramuscular, subcutaneous and intramedullary injections as well as intrathecal, direct intraventricular, intravenous, intraperitoneal, intranasal, or intraocular injections.

Alternately, one may administer the pharmaceutical composition in a local rather than systemic manner, for example, via injection of the pharmaceutical composition directly into the tumor (i.e. in situ).

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of the present invention may also be formulated in rectal compositions such as suppositories or retention enemas, using, e.g., conventional suppository bases such as cocoa butter or other glycerides.

Pharmaceutical compositions suitable for use in context of the present invention include compositions wherein the active ingredients are contained in an amount effective to achieve the intended purpose. More specifically, a therapeutically effective amount means an amount of active ingredients (naked silencing oliginucleotides, nucleic acid construct comprising same and/or encapsulated or shielded silencing oliginucleotides and constructs comprising same) effective in reducing VDAC1 expression and in preventing, alleviating or ameliorating tumor progression, invasion and recurrence, or prolonging the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”. Ch. 1 p.1).

Dosage amount and interval may be adjusted individually to provide plasma or brain levels of the active ingredient are sufficient to induce or suppress the biological effect (minmmal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as if further detailed above.

The following examples are presented in order to more fully illustrate some embodiments of the invention. They should, in no way be construed, however, as limiting the broad scope of the invention. One skilled in the art can readily devise many variations and modifications of the principles disclosed herein without departing from the scope of the invention.

### EXAMPLES

### Material and Methods

### Materials

The cell transfection agents JetPRIME and JetPEI were from PolyPlus transfection (Illkirch, France), while non-modified and 2′-O-methyl-modified hVDAC1-siRNAs were obtained from Genepharma (Suzhou, China). Polyethylenimine (PEI), Poly(D,L-lactide-coglycolide) (PLGA), Polyvinyl alcohol (PVA), propidium iodide (PI), sulforhodamine B (SRB), Tween-20, hematoxylin and eosin were obtained from Sigma (St. Louis, MO). Paraformaldehyde was purchased from Emsdiasum (Hatfield, PA). Dulbecco's modified Eagle's medium (DMEM) and Roswell Park Memorial Institute (RPMI) 1640 growth media were obtained from Gibco (Grand Island, NY). Normal goat serum (NGS) and the supplements fetal calf serum (FCS), L-glutamine and penicillin/streptomycin were obtained from Biological Industries (Beit Haemek, Israel). A cancer stem cell TF activation profiling plate array was obtained from Signosis (Santa Clara, CA). Primary antibodies, their source and the dilution used are detailed in Table 1. Horseradish peroxidase (HRP)-conjugated anti-mouse, anti-rabbit and anti-goat antibodies were from KPL (Gaithersburg, MD). TUNEL stain was obtained from Promega (Madison, WI).

### Cell Culture and Transfection

U-87MG, U-118, LN-18 (human glioblastoma), MZ-18 and MZ327 (human glioblastoma-patient derived cell line, and GL-261 (mouse glioblastoma) cells were maintained in DMEM culture medium supplemented with 10% FBS (5% for L18), 1 mM Lglutamine, 100 U/ml penicillin, and 100 μg/ml streptomycin. U-251 (human glioblastoma) cells cultured in RPMI-1640 medium supplemented with 10% FBS, 1 mM L-glutamine, 100 U/ml penicillin, and 100 μg/ml streptomycin at 37° C.; and 5% CO₂. G7-glioma-derived stem cell line were grown using specific glioblastoma stem cell medium, as described (Pollard S M et al. 2009. Cell Stem Cell. 4(6), 568-580). Non-modified and 2′-O-methylmodified siRNAs targeted to hVDAC1 (si-hVDAC1) were synthesized by Dharmacon or were obtained from Genepharma.

The following sequences were used:
Human VDAC1 silencing siRNA (si-hVDAC1):
si-hVDAC1: Sense: 5′ACACUAGGCACCGAGAUUA3′ (SEQ ID NO:3, positions 238-256 of VDAC1 coding sequence (SEQ ID NO:2); and
Antisense: 5′UAAUCUCGGUGCCUAGUGU3′ (SEQ ID NO:94)
si-hVDAC1 2/A: Sense: 5′ACACUAGGCACCGAGAUUA3′- with 2′-O-methyl-modified nucleotides indicated in bold and underlined (SEQ ID NO:14); and
Antisense: 5′UAAUCUCGGUGCCUAGUGU3′ with 2′-O-methyl-modified nucleotides indicated in bold and underlined (SEQ ID NO:15).
Scrambled siRNA (si-Scr) sequences:
Scrambled-Sense: −5′GCAAACAUCCCAGAGGUAU3′ with 2′-O-methyl-modified nucleotides indicated in bold and underlined (SEQ ID NO:24) and
Antisense: −5′AUACCUCUGGGAUGUUUGC3′ with 2-O-methyl-modified nucleotides indicated in bold and underlined (SEQ ID NO:25).
Addition silencing siRNA specific to human as well as murine VDAC1 was also designed:
VDAC1M/H, Sense: 5′-GAAUAGCAGCCAAGUAUCAG-3′ (SEQ ID NO:10, positions 487-505 of VDAC1 coding sequence (SEQ ID NO:2), flanked by tt nucleotides; and
Antisense: 5′-UGAUACUUGGCUGCUAUUC-3′ (SEQ ID NO:13) flanked by tt nucleotides.

Cells were seeded (150,000 cells/well) on 6-well culture dishes to 40-60% confluence and transfected with 10-100 nM si-Scr or si-hVDAC1 using the JetPRIME transfection reagent (Illkirch, France), according to the manufacturers' instructions.

### Xenograft and Intracranial-Orthotopic Xenograft Mouse Model

U-87MG (2×10⁶) and U-118MG (3×10⁶) glioblastoma cells were inoculated s.c. into the hind leg flanks of athymic eight-week old male nude mice (Harlan). Eleven days post-inoculation, tumor volume was measured (50-80 mm³) and mice were randomized into two groups (9 animals/group), treated with si-Scr or si-hVDAC1 mixed with in vivo JetPEI reagent and injected into the established s.c. tumors (50 nM final, 2 boluses) every three days. At the end of the experiments, the mice were sacrificed, tumors were excised, and half of each tumor was either fixed and processed for IHC or frozen in liquid nitrogen for later immunoblot and RNA isolation.

For the intracranial-orthotopic mouse model, U-87MG cells were treated with 50 nM si-Scr or si-hVDAC1 twice at a 4-day interval and engrafted into a nude mouse brain using a stereotactic device. The anesthetized mice were immobilized in a stereotactic-head frame (Stoelting, Wood Dale, IL) and a middle incision was made on the skull and a burr hole placed 0.5 mm anterior to the bregma and 2.5 mm lateral to the midline using a drill (Stoelting). A 31-gauge needle (Hamilton syringe) was used to deliver tumor cells. The needle tip was inserted into the brain 3 mm deep, relative to the skull surface, and maintained at this depth for 2 minutes before injection of tumor cells. Under sterile conditions, a 3 μL solution containing U-87MG (8×10⁴) in phosphate buffered saline (PBS) cells were injected into the brain parenchyma over a period of 3 minutes using and UltraMicroPump III (World Precision Instruments, Sarasota, FL). After infusion, the needle was left in place for 1 minute before slow withdrawal. The burr hole was sealed using sterile bone wax, and the wound was closed with 5.0 nylon surgical suture. All surgical procedures were performed under sterile conditions. Tumor development was monitored using MRI 22 and 33 days after cell engrafting. At the end of the experiment, mice were sacrificed, and their brains were excised and processed for IHC.

In vivo brain MRI was performed using the M7 1-Tesla compact ICON system (Aspect Imaging, M7, Israel), equipped with a set of application-specific radiofrequency (RF) 20-mm mouse head coils. For in vivo imaging, animals were maintained in an anesthetized state with 1.5% isoflorane in O₂ and placed on a specially designed heated bed where physiological signals, such as breath rate, were monitored throughout the experiment to ensure the animals' well-being. MRI acquisition parameters included fast spin echo with a repetition time of 2,500 ms and echo time of 74 m. Fifteen axial slices of 0.4 mm with a gap of 0.1 and a matrix of 256×256, field of view of 40 mm and acquisition time of 14.3 were collected (Tempel-Brami C et al. 2015. Toxicol Pathol 43, 633-650). Data were analyzed VivoQuant 2.10 software. The experimental protocols used were approved n the Institutional Animal Care and Use Committee.

### Immunoblot

For immunostaining, membranes containing electro-transferred proteins following SDS-PAGE were blocked with 5% non-fat dry milk and 0.1% Tween-20) in TBS, incubated with the primary antibodies (sources and dilutions as detailed in Table 2) and then with HRP-conjugated anti-mouse or anti-rabbit (1:10,000) or anti-goat (1:20,000) IgG. Enhanced chemiluminiscent substrate (Pierce Chemical. Rockford, IL) was used for detection of HRP activity.

TABLE 2
Antibodies against the indicated protein, their catalogue number,
source and the dilutions used in IHC, immunoblot (WB) and
immunofluorescence (IF) experiments

| | | Dilution | | |
|---|---|---|---|---|
| Antibody | Source and Cat. No. | IHC | WB | IF |
| Mouse monoclonal | Millipore, Billerica, MA, | — | 1:10000 | — |
| anti-actin | MAB1501 | | | |
| Mouse monoclonal | Abcam, Cambridge, UK, | 1:300 | 1:1000 | — |
| anti-ATP5a | ab14748 | | | |
| Mouse monoclonal | Abcam, Cambridge, UK | 1:100 | 1:1000 | 1:200 |
| anti-β III tubulin | ab7751 | | | |
| Rabbit polyclonal | Abcam, Cambridge, UK | 1:50 | | — |
| anti-CD31 | ab28364 | | | |
| Rabbit polyclonal | Abcam, Cambridge, UK | 1:1000 | 1:3000 | — |
| anti-CD44 | ab157107 | | | |
| Mouse monoclonal | Miltenyi Biotec GmbH, | — | 1:1500 | — |
| anti-CD133 | AC133 | | | |
| Rabbit polyclonal | Abcam, Cambridge, UK | 1:200 | 1:4000 | — |
| anti-citrate synthase | ab96600 | | | |
| Rabbit polyclonal | Dako, CL, USA, A4502 | 1:400 | — | — |
| anti c-Kit | | | | |
| Mouse monoclonal | BD Bioscience, San Jose, | 1:400 | 1:2000 | — |
| anti-cytochrome c | CA, 556432 | | | |
| Rabbit monoclonal | Abcam, Cambridge, UK, | 1:200 | 1:2000 | — |
| cytochrome c | ab150422 | | | |
| oxidase subunit VIc | | | | |
| Rabbit polyclonal | Abcam, Cambridge, UK, | 1:300 | 1:2000 | — |
| anti EGFR | ab2430 | | | |
| Mouse monoclonal | Invitrogen, Life | 1:100 | 1:1000 | — |
| anti-E-cadherin | Technologies, NY, 18- | | | |
| | 0223 | | | |
| Rat monoclonal | Santa Cruz | 1:150 | — | — |
| anti-F4/80 | Biotechnology, Inc. | | | |
| | Dallas, TX, sc52664 | | | |
| Rabbit polyclonal | Abcam, Cambridge, UK, | 1:300 | 1:1500 | — |
| anti-FoxP1 | ab191184 | | | |
| Mouse monoclonal | Abcam, Cambridge, UK, | 1:200 | 1:1000 | — |
| anti-GAPDH | ab9484 | | | |
| Mouse monoclonal | Abcam, Cambridge, UK, | — | 1:2000 | 1:1500 |
| anti-GAD67 | ab26116 | | | |
| Mouse monoclonal | Santa Cruz | 1:200 | 1:1000 | 1:150 |
| anti-GFAP | Biotechnology, Inc. | | | |
| | Dallas, TX, sc-33673 | | | |
| Rabbit monoclonal | Abcam, Cambridge, UK | 1:200 | 1:1500 | — |
| anti-Glut1 | ab40084 | | | |
| Mouse monoclonal | Abcam, Cambridge, UK | 1:500 | 1:2000 | — |
| anti-HK-I | ab105213 | | | |
| Rabbit polyclonal | Abcam, Cambridge, UK | 1:400 | 1:2000 | — |
| anti-HK II | ab3279 | | | |
| Rabbit polyclonal | Abcam, Cambridge, UK, | 1:300 | 1:1000 | — |
| ant-HNF4 | ab92378 | | | |
| Rabbit monoclonal | Thermo Scientific, NY | 1:100 | — | — |
| anti-Ki67 | RM-9106-s1 | | | |
| Rabbit polyclonal | IMGENX Littleton, | 1:200 | 1:1000 | — |
| anti-KLF4 | USA, IMG-6081-A | | | |
| Rabbit monoclonal | Epitomics, Cambridge, | 1:300 | 1:1000 | — |
| anti-LDH | UK, 1980-1 | | | |
| Goat polyclonal | Santa Cruz | — | 1:1500 | — |
| anti-LDH-A | Biotechnology, Inc. | | | |
| | Dallas, TX, sc-27230 | | | |
| Mouse monoclonal | Sigma-Aldrich, St. Louis, | 1:400 | — | |
| anti-Map2 | MI, M 4403 | | | |
| Rabbit polyclonal | Abcam, Cambridge, UK, | 1:300 | — | — |
| anti-Maz | ab114965 | | | |
| Mouse monoclonal | Millipore, Billerica, MA, | 1:200 | — | — |
| anti-Musashi-1 | MABE268 | | | |
| Mouse monoclonal | Invitrogen (Life | 1:200 | — | — |
| anti-N-cadherin | Technologies), NY | | | |
| | 18-0224 | | | |
| Rabbit polyclonal | Millipore, Billerica, MA, | 1:400 | 1:1000 | 1:1500 |
| anti-Nestin | MAB353 | | | |
| Rabbit polyclonal | Santa Cruz | 1:200 | 1:1000 | — |
| anti-NGFR | Biotechnology, Inc. | | | |
| | Dallas, TX, sc-8317 | | | |
| Rabbit polyclonal | Abcam, Cambridge, UK, | 1:200 | — | — |
| anti NRSF/Rest | ab70300 | | | |
| Mouse monoclonal | Santa Cruz | — | 1:5000 | — |
| anti-P53 | Biotechnology, Inc., | | | |
| | Dallas, TX, sc-126 | | | |
| Goat polyclonal | Millipore, Billerica, MA, | 1:300 | 1:2000 | — |
| anti-S100b | ABN59 | | | |
| Goat polyclonal | Santa Cruz | 1:200 | 1:1500 | — |
| anti-Sox2 | Biotechnology, Inc. | | | |
| | Dallas, TX, sc-17320 | | | |
| Rabbit monoclonal | Abcam, Cambridge, UK, | 1:500 | 1:5000 | — |
| anti-VDAC1 | ab154856 | | | |
| Mouse monoclonal | Santa Cruz | 1:50 | 1:1000 | — |
| anti-VEGF | Biotechnology, Inc. | | | |
| | Dallas, TX. sc-65617 | | | |
| Mouse monoclonal | Invitrogen, Life | 1:300 | 1:1000 | — |
| anti-Vimentin | Technologies, NY | | | |
| | 18-0052 | | | |

### IHC and Immunofluorescence of Tumor Tissue Sections

Immunohistochemical (IHC) and immunofluorescence staining was performed on 5 μm-thick formalin-fixed and paraffin-embedded tumor tissue sections. The sections were deparaffinized by placing the slides at 60° C., for 1 h and using xylene. Thereafter, the tissue sections were rehydrated with a graded ethanol series (1000%-50%). Antigen retrieval for some proteins (VDAC1, Glut1, citrate synthases, HK-II, GAPDH, LDH, Nestin, NGFR, KLF4, Sox2, S100b, Mushahi, Map2, b-III tubulin (TUBB3), GAD67, ATP5a, cytochrome c oxidase subunit Vic, c-Kit, N-cadherin, E-cadherin and vimintein) was performed in 0.01 M citrate buffer (pH 6.0). For HK-I and CD31, VEGF, Ki-67, GFAP, F4/80, HNF4, FOXP1, NRSF/Rest and MAZ, antigen retrieval was performed in 10 mM Tris-EDTA (pH 9) and 0.5 M Tris (pH 10), for 30 minutes each at 95-98° C. After washing sections in PBS containing 0.1% Triton-X100 (pH 7.4), non-specific antibody binding was reduced by incubating the sections in 10% NGS for 2 h. After decanting excess serum, sections were incubated overnight at 4° C. with primary antibodies (sources and dilutions used detailed in Table 1). Sections were washed with PBST. For IHC, endogenous peroxidase activity was blocked by incubating the sections in 3% H₂O₂ for 15 min. After washing thoroughly with PBST, the sections were incubated for 2 h with the appropriate secondary antibodies. For IHC, anti-mouse or anti rabbit (1:250) secondary antibodies conjugated to HRP were used, as appropriate. Sections were washed five times in PBST and the peroxidase reaction was subsequently visualized by incubating with 3.3-diaminobenzidine (DAB) (ImmPact-DAB, Burlingame, CA). After rinsing in water, the sections were counterstained with hematoxylin and mounted with mounting medium. Finally, the sections were observed under a microscope (Leica DM2500) and images were collected at 20× magnification with the same light intensity and exposure time. Non-specific control experiments were carried out using the same protocols but omitting incubation with the primary antibodies. Hematoxylin-eosin staining was performed as described previously (Kiernan J. 2008. Histological and Histochemical Methods; Theory and Practice, 4th edition). For immunofluorescence. Cy3-conjugated anti-rabbit (1:500) or Cy2-conjugated anti-mouse (1:200) secondary antibodies were used. The cells were then stained with DAPI (0.07 μg/ml) and viewed with an Olympus IX81 confocal microscope.

### TUNEL Assay

Fixed tumor sections in paraffin were processed for the TUNEL assay using the DeadEnd Fluorometric TUNEL system (Promega, Madison, WI) according to the manufacturer's instructions. Sections were deparaffinized, equilibrated in PBS, permeabilized with proteinase K (20 μg/gml in PBS), post-fixed in 4% paraformaldehyde, and incubated in TdT reaction mix (Promega) for 1 h at 37° C. in the dark. Slides were then washed in 2× saline-sodium citrate (SSC) buffer and counter-stained with propidium iodide (1 μg/ml), and cover slipped with Vectashield mounting medium (Vector Laboratories. Burlingame. CA). Fluorescent images of apoptotic cells (green) and cell nuclei (red) were captured using a confocal microscope (Olympus 1X81).

### RNA Preparation and DNA Microarray Analysis

Total RNA was isolated from si-Scr- and si-hVDAC1-treated tumors (2 mice each) using an RNeasy mini kit (Qiagen) according to the manufacturer's instructions. Total RNA quality was analyzed using the Agilent RNA 6000 nano kit. The RNA integrity values obtained for total RNA extracted from si-Scr- and si-hVDAC1-treated tumors were 8-10 and 7-8.5, respectively. The targets for Affymetrix whole transcript expression microarray analyses were prepared using the Affymetrix GeneChip WT PLUS reagent kit according to the manufacturer's instructions and hybridized to Human Gene 1.0 ST microarrays. Data were acquired using the Affymetrix GeneChip algorithm (version 3.2). CEL files were imported to Partek® Genomics Suite® and all probes except control probes were preprocessed by RMA background correction, log 2 transformation and probeset summarization using median polish. Probesets having signal <5 in all samples were filtered out. Subsequently, global scaling was carried out by shifting the mean of each sample to the grand mean (i.e. in each sample, the mean signal was subtracted from each of the signals, and then the grand mean from all samples was added, such that all arrays eventually had the same mean signal). Differentially expressed genes were defined as those having FDR-adjusted t-test p-value <0.05, and two clusters were defined: upregulated and downregulated genes (linear fold change >2 and <−2, respectively). Each cluster was tested separately for enrichment of functional groups based on the GO system (Ashburner M et al., 2000. Nature genetics 25, 25-29.). Promoter analysis was performed using the DAVID (Huang et al., 200)) and Expander (Ulitsky I et al., 2010. Nature protocols 5, 303-322) software tools.

Quantitative Real-Time PCR (qRT-PCR)

Total RNA was isolated as described above. Complementary DNA was synthesized from 1 μg total RNA using a Verso cDNA synthesis kit (Thermo Scientific). Real-tie fluorescent RT-PCR was performed using specific primers (KiCqStaT Primers; Sigma Aldrich) in triplicate, using Power SYBER green master mix (Applied Biosystems, Foster City, CA). The levels of the target genes were normalized relative to β-actin mRNA levels. Samples were amplified by a 7300 Real Time PCR System, (Applied Biosystems) for 40 cycles using the following PCR parameters: 95° C. for 15 seconds, 60° C. for minute, and 72° C. for 1 minute. The copy numbers for each sample were calculated by the CT-based calibrated standard curve method. The mean fold changes (±SEM) of the three replicates were calculated. Primers designed for gene expression examination are listed in Table 3.

TABLE 3
Real-Time PCR Primers Used in this Study, Related to Experimental
Procedures

| Gene | Primer sequences | SEQ ID NO: |
|---|---|---|
| β-Actin | Forward 5′-ACTCTTCCAGCCTTCCTTCC-3′ | 26 |
| | Reverse 5′-TGTTGGCGTACAGGTCTTTG-3′ | 27 |
| ALDH1L1 | Forward 5′-CCAAAGTCCTGGAGGTTGAA-3′ | 28 |
| | Reverse 5′-TAACTCCAGGCCATCACACA-3′ | 29 |
| BLPB (FABP7) | Forward 5′-AGTTTCCAGCTGGGAGAAGAG-3′ | 30 |
| | Reverse 5′-CTTTGCCATCCCATTTCTGTA-3′ | 31 |
| CAST | Forward 5′-CCAGAACCTATGCTGGTGGA-3′ | 32 |
| | Reverse 5′-GGCTTTTTGGCTTGGTTGACT-3′ | 33 |
| CS | Forward 5′-AGGAACAGGTATCTTGGCTCT-3′ | 34 |
| | Reverse 5′-GGGGTGTAGATTGGTGGGAA-3′ | 35 |
| c-Myc | Forward 5′-GTAGTGGAAAACCAGCAGCC-3′ | 36 |
| | Reverse 5′-CCTCCTCGTCGCAGTAGAAA-3′ | 37 |
| DLST | Forward 5′-TGCAAGGATGACTTGGTTACAG-3′ | 38 |
| | Reverse 5′-CTTCTGCAACTGTGTCTCCAAC-3′ | 39 |
| GFAP | Forward 5′-AAGCTCCAGGATGAAACCAAC-3′ | 40 |
| | Reverse 5′-AGCGACTCAATCTTCCTCTCC-3′ | 41 |
| GAPDH | Forward 5′-TGGAAGGACTCATGACCACA-3′ | 42 |
| | Reverse 5′-ATGATGTTCTGGAGAGCCCC-3′ | 43 |
| GLUT1 | Forward 5′-GGCCATCTTTTCTGTTGGGG-3′ | 44 |
| | Reverse 5′-TCAGCATTGAATTCCGCCG-3′ | 45 |
| Hif-1 α | Forward 5′-CTGACCCTGCACTCAATCAA-3′ | 46 |
| | Reverse 5′-TCCATCGGAAGGACTAGGTG-3′ | 47 |
| HK-1 | Forward 5′-GTCTCAGTCCAGCACGTTTG-3′ | 48 |
| | Reverse 5′-GAAACGCCGGGAATACTGTG-3′ | 49 |
| IDH3a | Forward 5′-CCACATGGGACTTTTTGACC-3′ | 50 |
| | Reverse 5′-TTACTCGGCGACAGATTTCC-3′ | 51 |
| Ki-67 | Forward 5′-CTTTGGGTGCGACTTGACG-3′ | 52 |
| | Reverse 5′-GTCGACCCCGCTCCTTTT-3′ | 53 |
| LDH-A | Forward 5′-GCAGGTGGTTGAGAGTGCTT-3′ | 54 |
| | Reverse 5′-GCACCCGCCTAAGATTCTTC-3′ | 55 |
| MAP2 | Forward 5′-TCCAAAATCGGATCAACAGAC-3′ | 56 |
| | Reverse 5′-AGAGCCACATTTGGATGTCAC-3′ | 57 |
| Nanog | Forward 5′-TGGGATTTACAGGCGTGAGCCAC-3′ | 58 |
| | Reverse 5′-AAGCAAAGCCTCCCAATCCCAAAC-3′ | 59 |
| N-Cadherin | Forward 5′-AGGGATCAAAGCCTGGAACA-3′ | 60 |
| | Reverse 5′-TTGGAGCCTGAGACACGATT-3′ | 61 |
| Nestin | Forward 5′-GAAACAGCCATAGAGGGCAAA-3′ | 62 |
| | Reverse 5′-TGGTTTTCCAGAGTCTTCAGTGA-3′ | 63 |
| Oct-3/4 | Forward 5′-GGGCTCTCCCATGCATTCAAAC-3′ | 64 |
| | Reverse 5′-CACCTTCCCTCCAACCAGTTGC-3′ | 65 |
| PCNA | Forward 5′-GCCGAGATCTCAGCCATATT-3′ | 66 |
| | Reverse 5′-ATGTACTTAGAGGTACAAAT-3′ | 67 |
| PTPN12 | Forward 5′-TGACAGAGGAACGGGGGTAT-3′ | 68 |
| | Reverse 5′-CGGCTGTGATCAAATGGCAG-3′ | 69 |
| Sanil1 | Forward 5′-AGTGGTTCTTCTGCGCTACT-3′ | 70 |
| | Reverse 5′-CTGCTGGAAGGTAAACTCTGG-3′ | 71 |
| Snail2 | Forward 5′-ACATACAGTGATTATTTCCCCGT-3′ | 72 |
| | Reverse 5′-CGCCCCAAAGATGAGGAGTA-3′ | 73 |
| SOX10 | Forward 5′-AGGAGAGGTCCGAGGAGGTG-3′ | 74 |
| | Reverse 5′-CTCAGCTCCACCTCCGATAG-3′ | 75 |
| SOX2 | Forward 5′-CCATGCAGGTTGACACCGTTG-3′ | 76 |
| | Reverse 5′-TCGGCAGACTGATTCAAATAA-3′ | 77 |
| SSEA-1 | Forward 5′-TGAAATAGCTTAGCGGCAAGA-3′ | 78 |
| | Reverse 5′-GTGAATCGGGAACAGTTGTGT-3′ | 79 |
| SUCLG2 | Forward 5′-AAGAGGTGGCTGCTTCAAAC-3′ | 80 |
| | Reverse 5′-AGCCTAGATTTTCGGCCATC-3′ | 81 |
| Tnc | Forward 5′-ATGAATCAGTGGATGGCACA-3′ | 82 |
| | Reverse 5′-CCATTGAGTGCCTGGATCTT-3′ | 83 |
| TUBB3 | Forward 5′-CTCAGGGGCCTTTGGACATC-3′ | 84 |
| | Reverse 5′-CAGGCAGTCGCAGTTTTCAC-3′ | 85 |
| Twist | Forward 5′-TCTTACGAGGAGCTGCAGAC-3′ | 86 |
| | Reverse 5′-TATC CAGCTCCAGAGTCTCT-3′ | 87 |
| Zeb1 | Forward 5′-TTCCTGAGGCACCTGAAGAG-3′ | 88 |
| | Reverse 5′-GGTGTTCCATTTTCATCATGACC-3′ | 89 |
| Zeb2 | Forward 5′-TAGTGTGCCCAACCATGAGT-3′ | 90 |
| | Reverse 5′-TTGCATTCTTCACTGGACCA-3′ | 91 |
| p53 | Forward 5′-AGGTTGGCTCTGACTGTACC-3′ | 92 |
| | Reverse 5′-AAAGCTGTTCCGTCCCAGTA-3 | 93 |

### Transcription Factors (TF) Profiling Array

Nuclear extracts were prepared from si-Scr- or si-hVDAC1 t-treated tumors using a Nuclear/cytosol fractionation kit (Biovision, Milpitas, CA) following the manufacturer's instructions. TF DNA-binding activity was analyzed as per the manufacturer's instructions (Signosis, Sunnyvale, CA). Briefly, a nuclear extract (˜13 μg of protein) was added to a mixture of DNA sequences encoding 23 different TF-binding sites (BS) and incubated at 16° C. for 30 min. The samples containing the formed TF-DNA complexes were then separated from free DNA probes using an isolation column, with an aliquot (100 μl) being applied to the column and TF-DNA complexes being eluted with elution buffer (200 μl). A sample (95 μl) was then added to each well of a 96-well plate contained an immobilized complementary sequence to one of the 23 TFs. The plate was sealed and incubated overnight at 42° C. for hybridization. The plate was then washed three times, and developed using streptavidin conjugated-HRP and luminescence obtained following substrate addition was recorded using an Infinite M1000 microplate reader.

PLGA Encapsulation of siRNA

VDAC1 siRNA-loaded PEI-PLGA complexes were prepared by the solvent displacement method with some modifications, as previously reported (Fessi H et al., 1989. Int. J. Pharm. 55, 1-4; Das J et al., 2014. Toxicol Lett, 225, 454-66). A pre-formed complex of PEI (20 mg) dissolved in 1% of polyvinyl alcohol (PVA) and siRNA 360 μl siRNA solution (50 μM) was incubated for 30 min at 37° C. PLGA (50 mg) was dissolved in 1 ml acetone. To this organic mixture, 10 mil of aqueous solution containing 1% PVA (w/v) stabilizer was added in a drop-wise manner (0.5 ml/min). Subsequently, the pre-formed complexes containing siRNAs were added in a drop-wise manner. The mixtures were stirred continuously at room temperature until complete evaporation of the organic solvent. The nanoparticles were centrifuged at 15000 g (4° C. for 30 min) and the pellet was re-suspended in DEPC-water, washed three times and the resulting nanoparticles containing the siRNA-loaded suspension was stored at −20° C. until further use.

### Neurosphere Formation

Conditions for neurosphere formation were as described previously (Rota L M et al. 2012. J Mammary Gland Biol Neoplasia 17: 119-123. Briefly, U-87MG, G7, MZ-18 and MZ-327 cells were treated with si-Scr or si-hVDAC1 (50 nM) and 24 h post-transfection, the cells (10⁵) were cultured in 12-well plates coated with a soft agar (5%) layer prepared in neuronal stem cell medium, as described previously (Pollard et al. 2009, tbid) Following 24 h incubation at 37° C. in a 5% CO₂ atmosphere, the cultures were photographed.

### Statistics and Data Analysis

The data was shown as the mean±SEM, of at least three independent experiments unless specified differently. Significance of differences was calculated by the two-tailed Student's t-test using the T Test function provided by Microsoft Excel. Statistically significance is reported at p<0.05 (*), p<0.01 (**) or p<=0.001 (***). For survival analysis, Kaplan-Meter plots were used.

### Example 1: VDAC1 Depletion Induces Changes in Cell Morphology and Expression of Differentiation Associated Proteins

The levels of several differentiation markers in si-Scr and si-hVDAC1-treated tumors (TTs) were examined. As GBM is often considered a grade IV astrocytoma in which astrocytes lack normally recognizable features, human normal brain and GBM sections were hematoxylin and eosin (H&E) stained Normal brain showed astrocytes, a few small vessels and mainly fibrillary stroma (FIG. 1A). In contrast, GBM sections showed large irregular cells with atypical nuclei (FIG. 1B). Staining of normal brain for GFAP (glial fibrillary acidic protein), an intermediate filament protein expressed by central nervous system cells, including astrocytes, showed cells with long and marked processes (FIG. 1A). GFAP-staining of GBM sections showed cells with no processes, emphasizing the highly malignant nature of the cancer (FIG. 1B).

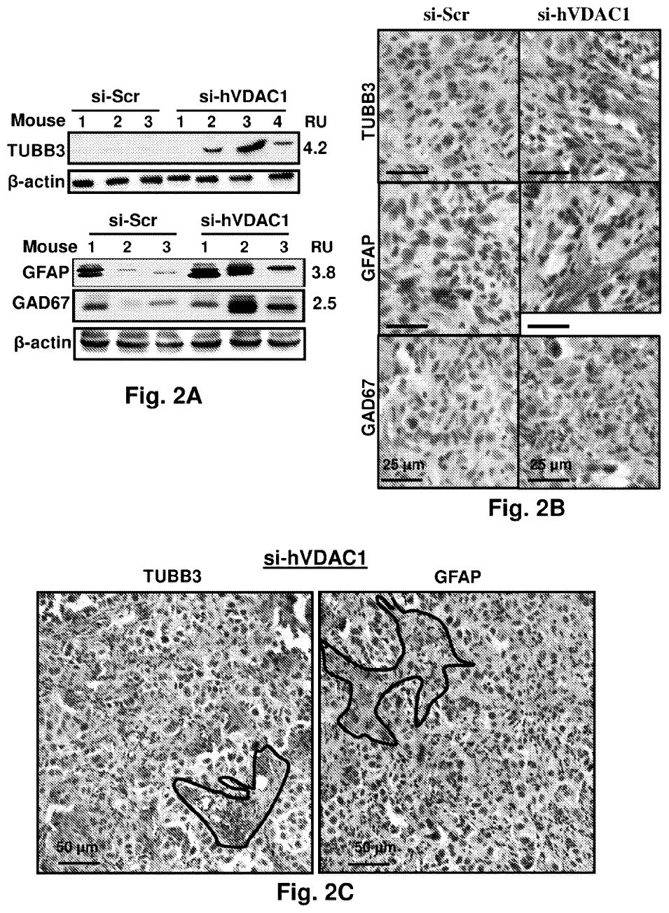

H&E-stained si-Scr-TT sections revealed cells with large nuclei (FIG. 1D), similar to those seen in GBM patients (FIG. 1B). In contrast, si-hVDAC1-TTs showed astrocyte-like cell morphology with long and marked processes (FIG. 1C), resembling normal brain tissue (FIG. 1A), with increased GFAP staining. Next immunostaining for the mature neuron markers, such as microtubule-associated protein 2 (MAP-2) and tubulin beta-III (TUBB3) (FIG. 1E, H and FIG. 2 A-C) was performed. Both were highly expressed in the si-hVDAC1-TTs. Increased GFAP and TUBB3 expression in the si-hVDAC1-TTs was also demonstrated by Western blotting (WB) (FIG. 1F). Similar results were obtained by qRT-PCR (FIG. 1G). These results indicate that si-hVDAC1 treatment induced morphological changes in tumor cells to resemble normal brain cells, and altered expression of neuronal markers, suggesting differentiation of tumor cells into astrocytes and neuron-like cells. To verify possible cell differentiation stages (Wiese S et al., 2012. Frontiers in pharmacology 3, 120) of si-hVDAC1-TTs, markers of cell differentiation stages were screened using qRT-PCR. Reductions (4-120-fold) in the levels of markers for early and late precursor cells, of immature astrocytes and neurons, and of the neuronal precursor and progenitor marker nestin, brain lipid binding protein (BLBP), stage-specific embryonic antigen 1 (SSEA1/CD15), tenascin C (TNC), and aldehyde dehydrogenase 1 L1 (ALDH1L1) was observed. At the same time, increase (15-120-fold) in the levels of markers associated with mature astrocytes and neurons (GFAP, TUBB3 and MAP2) were found (FIG. IG).

These findings were further supported by tumor section immunofluorescent staining. Nestin staining was highly decreased, while TUBB3, GFAP, and glutamate decarboxylase 1 (GAD1/GAD67), involved in GABA synthesis, were strongly stained (FIG. 1H). Similar results were obtained with U-118 cells (FIG. 2). The results indicate that GBM expressed precursor cells are capable of differentiation into neuronal lineages and that si-hVDAC1 tumor treatment led cells to differentiate towards mature astrocytes and neuron-like cells.

### Example 2: VDAC1 Depletion Alters Transcription Factor (T) Expression Profiles

To better understand the molecular mechanism underlying cell signaling and gene expression altered by treating the tumor with si-hVDAC1, shown to be leading to cell differentiation, changes in the expression levels of transcription factors (TFs) in the nuclear fraction were screened for. Cancer stem cell TF activation profiling plate array was used (FIG. 3A), si-hVDAC1-TTs expressed increased levels of tumor suppressor TFs, such as p53, Forkhead box O3 (FoxO3), PR domain-containing 14 (PRDM14) and signal transducer and activator of transcription 3 (STAT3). Expression levels of the major TFs p53, RIF-1α and c-Myc, known to regulate metabolism, cell growth, proliferation and differentiation (Yeung S Jet al. 2008. Cellular and molecular life sciences: CMLS 65, 3981-3999) were significantly affected by si-hVDAC1 treatment. p53 levels were elevated in si-hVDAC1-TTs, while the expression levels of HIF-1α and c-Myc were reduced, as revealed by TF activation profiling assay, qRT-PCR, and immunoblotting (FIG. 3A-D). The level of AP-2, involved in tumorigenesis and possibly acting as a tumor suppressor, was decreased in si-hVDAC-TTs (FIG. 3A).

Expression levels of the canonical major TFs p53, HIF-1α (hypoxia-inducible factor 1 alpha) and c-Myc, known to regulate metabolism, cell growth, proliferation and differentiation (Yeung S J et al., 2008. Cellular and molecular life sciences: CMLS 65, 3981-3999), were also significantly affected by si-hVDAC1 treatment. p53 levels were elevated in si-hVDAC1-TTs, while the expression levels of HIF-1α and c-Myc were reduced, as revealed by TF activation profiling assay, qRT-PCR, and WB (FIG. 3A-D).

The results (FIGS. 1, 3) thus present a complex set of effects of VDAC1 depletion on a network of key regulators of cell metabolism, leading to a reprogramming of cancer cells towards differentiation.

### Example 3: Tumor Treatment with si-hVDAC1 Eliminates Glioblastoma Stem Cells (GSCs)

The decrease in the levels of transcription factors (TFs) associated with stemness in the si-hVDAC1-TTs, such as Oct-3/4, AP1(Jun), Twist and Klf4 (FIG. 3A), and the increase in cell differentiation markers (FIG. 1) led to further analyses of the effects of VDAC1 depletion on the expression of GSCs-associated markers (FIG. 3E-H). Dynamic steadiness between self-renewing GSCs and differentiation is essential for GBM development (Bao et al., 2006) and invasion (FIG. 4A,B) (Singh et al., 2004). si-hVDAC1 treatment markedly decreased the expression of GSCs markers, such as CD133, Sox2, Klf4, Musashi, Nestin, NGFR, S100b and CD44, as evaluated by IHC and WB (FIG. 3E-G) and qRT-PCR, revealing decreases of several fold in Sox2, Oct3/4 and Nanog mRNA levels (FIG. 3H). Similar results were obtained with U-118 xenografts (FIG. 4C,D). The orthotopic tumors derived from si-Scr-treated U-87MG cells showed high levels of Nestin in patches (FIG. 3I), pointing to GSCs niches. No staining was obtained in brain injected with si-VDAC1 U-87MG-treated cells. Thus, si-VDAC1 tumor treatment resulted in apparent “disappearance” of GSCs (FIG. 3). Without wishing to be bound by any specific theory or mechanism of action, the reduction in the GSCs number is attributed to the promoted differentiation (FIG. 1). Arrest of cell proliferation caused by VDAC1 silencing may also contribute.

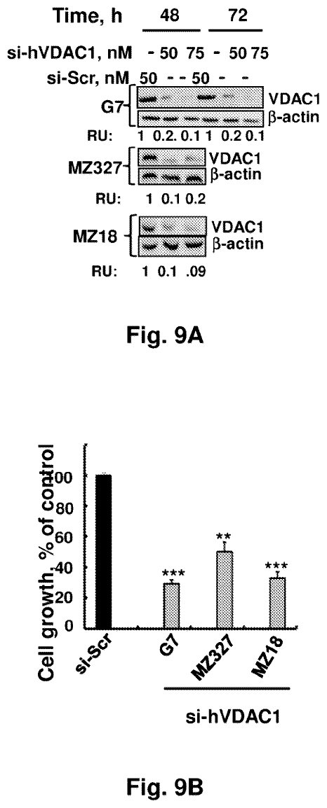

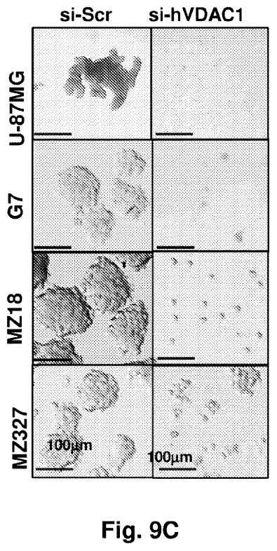

The effect of si-hVDAC1 treatment on stem cells was also demonstrated by analyzing neurosphere formation using neuro-stem cell medium. U-87MG, the glioma-derived G7 stem cell line and the human GBM patient-derived cell lines, MZ-18 and MZ-327 were treated with si-Scr or si-hVDAC1, showed decreased VDAC1 levels and inhibited cell growth (FIG. 9A-B). Neurospheres were formed in the si-Scr-treated but not in si-hVDAC1-treated cells (FIG. 9C). The reduced potential of neurosphere formation by reducing VDAC1 levels suggests a decrease in GSC levels upon metabolic reprograming.

### Example 4: Effect of hVDAC1 Silencing Epithelial to Mesenchymal Transition (EMT)

The tumor metastatic potential is attenuated by the epithelial to mesenchymal transition (EMT) (Mani SA et al., 2008. Cell 133, 704-715). To follow EMT, si-Scr- and si-hVDAC1-TTs were analyzed for the expression of epithelial and mesenchymal cell markers (FIG. 3J-L). IHC staining of si-hVDAC1-TTs showed a decrease in the mesenchymal cells markers N-cadherin (type I cell—cell adhesion glycoproteins) and vimentin (intermediate filaments) and an increase in E-cadherin, an epithelial cell marker (Agiostratidou G et al, 2007. Journal of mammary gland biology and neoplasia 12, 127-133), as compared to their levels in si-Scr-TTs (FIG. 3J). Similar results were obtained for E-cadherin and vimentin by WB (FIG. 3K) and qRT-PCR showing the mesenchymal markers Twist, Zeb1, Zeb2, Snail1, Snail 2, and N-cadherin (Barriere G et al., 2014. Annals of translational medicine 2, 109) to be decreased 3-12-fold in si-VDAC1-TTs (FIG. 3L). These results show that EMT was reversed in si-hVDAC1-TTs and that this attenuated the invasive potential of GBM cells (FIG. 4A,B).

### Example 5: Effect of hVDAC1 Silencing on Angiogenesis, Tumor-Associated Macrophage and Extracellular Matrix

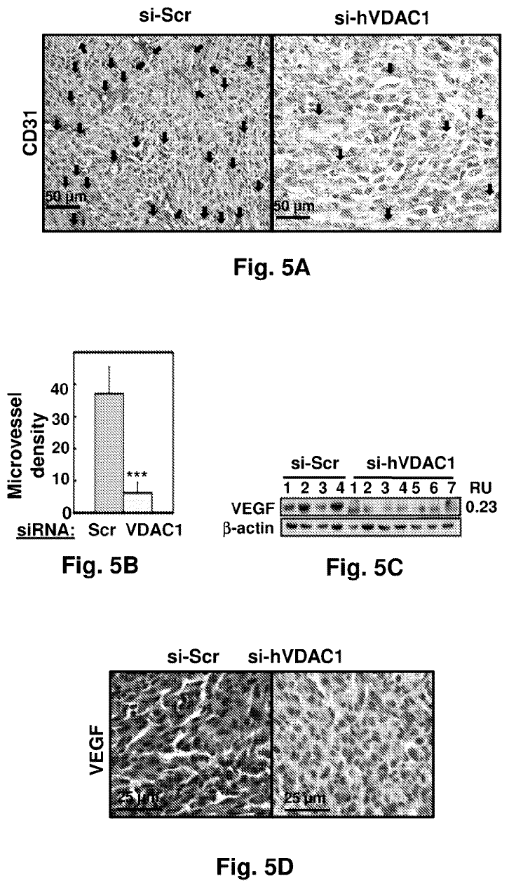

A solid tumor requires blood supply to continue growing and thus promotes angiogenesis, with GBM being highly angiogenic and invasive (Bai et al., 2011, ibid). Immunostaining of endothelial cells from si-hVDAC1-TT for CD31 showed a major decrease in the number of blood vessels, with quantitation of microvessel density (MVD) revealing a near 80% decrease (FIG. 5A,B).

In GBM, as in many other cancers, high levels of vascular endothelial growth factor (VEGF), activating highly expressed VEGF receptors in tumor endothelial vessels to stimulate angiogenesis, are expressed. VEGF level was dramatically decreased in si-hVDAC1-TTs (FIG. 5C, D). IHC analysis of si-hVDAC1-TTs also showed decreased levels of the tumor-associated macrophage specific marker, cell surface glycoprotein F4/80 (FIG. 4E). Similarly, DNA microarray analysis (Table 4) also showed that si-hVDAC1 treatment affected the expression of angiogenesis-, EMT- and invasion-related genes, reducing VEGF/A, TGFβ. Hif-1α, metalloproteinases, extracellular matrix and cell adhesion components, while increasing angiogenic and MMP inhibitors.

TABLE 4
Selected genes associated with tumor oncogenic properties differentially
expressed in si-Scr and si-hVDAC1-treated tumors, as identified by
DNA microarray analysis

| | si-hVDAC1- vs. | | |
|---|---|---|---|
| | si-Scr-treated | | |
| | tumors | | |
| Invasive related | Fold | | |
| genes | Change | p-value | Function |

MMP transcription and function inhibitors

| | | | |
|---|---|---|---|
| ID4-inhibitor of | 6.10 | 4.4E−04 | Transcriptional negative |
| DNA binding 4 | | | regulator of basic helix-loop- |
| | | | helix TFs |
| TIMP1- | −5.80 | 9.0E−05 | Inhibitor of several MMPs, |
| metallopeptidase | | | including MMP-3, MMP7-13 |
| inhibitor 1 | | | and MMP16 |
| TIMP2- | −4.00 | 3.4E−03 | Inhibitor of several MMPs, |
| metallopeptidase | | | including MMP-1-3, 7-10, |
| inhibitor 2 | | | 13-16, 19 |
| TIMP3- | −4.71 | 2.3E−03 | Inhibitor of several MMPs, |
| metallopeptidase | | | including MMP-1-3, 7, 9, |
| inhibitor 3 | | | 13-15 |

MMP proteins

| | | | |
|---|---|---|---|
| MMP2-matrix | −7.9 | 2.3E−03 | Metalloproteinase cleaving |
| metallopeptidase 2 | | | gelatin type I and collagen |
| | | | types IV, V, VII, X |
| MMP16-matrix | −2.3 | 7.9E−03 | Metalloproteinase cleaving |
| metallopeptidase 16 | | | collagen type III and |
| | | | fibronectin |
| ADAM9- | −11.0 | 5.0E−04 | Membrane-anchored zinc |
| Disintegrin and | | | protease implicated in cell-cell |
| metallo-proteinase | | | and cell-matrix interactions |
| domain-containing | | | |
| protein 9 | | | |
| ADAM12- | −7.1 | 6.0E−04 | Membrane-anchored zinc |
| Disintegrin and | | | protease involved in cell-cell |
| metallo-proteinase | | | and cell-matrix interactions |
| domain-containing | | | |
| protein 12 | | | |

ECM and integrin genes

| | | | |
|---|---|---|---|
| LAMB1-laminin, | −4.2 | 9.0E−03 | Component of the ECM |
| beta 1 chain | | | |
| LAMC1-laminin, | −5.1 | 6.6E−04 | Component of the ECM |
| gamma 1 | | | |
| ITGA2-integrin, | −4.9 | 2.6E−04 | Mediates cell matrix and cell- |
| alpha 2 | | | cell interactions |
| ITGA3-integrin, | −7.4 | 5.3E−04 | Mediates cell matrix and cell- |
| alpha 3 (subunit of | | | cell interactions |
| VLA-3) | | | |
| ITGA5-integrin, | −4.2 | 4.7E−03 | Mediates cell matrix and cell- |
| alpha 5 (fibronectin | | | cell interactions |
| receptor) | | | |
| ITGB1BP1-integrin | −4.2 | 3.7E−04 | Mediates cell matrix and cell- |
| beta 1 binding | | | cell interactions |
| protein 1 | | | |
| ITGB3BP-integrin | −6.6 | 1.2E−03 | Mediates cell matrix and cell- |
| beta 3 binding | | | cell interactions |
| protein | | | |
| CD44-Hyaluronic | −3.1 | 3.8E−04 | Receptor for hyaluronic acid |
| acid receptor | | | |
| CD99- | −9.0 | 2.1E−04 | Inhibits cell-extracellular |
| Transmembrane | | | matrix adhesion |
| glycoprotein | | | |
| CD164-Sialomucin | −21.2 | 2.1E−04 | Cell adhesion molecule |
| core protein 24 | | | |
| (endolyn) | | | |

Angiogenesis

| | | | |
|---|---|---|---|
| VEGFB-Vascular | 4.5 | 3.4E−04 | Growth factor for endothelial |
| endothelial growth | | | cells of newly formed blood |
| factor B | | | vessels |
| VEGFC-Vascular | −2.1 | 6.3E−05 | Growth factor for lymphatic |
| endothelial growth | | | endothelial cells |
| factor C | | | |
| NRP1-Neuropilin 1 | −4.7 | 1.8E−04 | Membrane co-receptor for both |
| | | | VEGF and Semaphorin, also |
| | | | functions in axon guidance, |
| | | | cell survival, migration, and |
| | | | invasion |
| TGFB1- | −3.1 | 2.3E−04 | Growth factor controlling |
| Transforming | | | proliferation, differentiation |
| growth factor, beta | | | and more cell functions |
| 1 | | | |
| TGFBR1- | −2.2 | 1.1E−03 | Co-receptor for TGFBR2 |
| Transforming | | | activated by bound TGFβ |
| growth factor, beta | | | |
| receptor 1 | | | |
| TGFBR2- | −6.7 | 1.5E−04 | Constitutively active co- |
| Transforming | | | receptor TGFBR1 of bound |
| growth factor, beta | | | TGFβ |
| receptor 2 | | | |
| CD109-tumor | −12.3 | 2.0E−04 | GPI-linked cell surface antigen |
| endothelial cells | | | |
| marker | | | |

Angiogenic inhibitors

| | | | |
|---|---|---|---|
| HIF1AN-hypoxia | −2.3 | 6.8E−04 | Preventing interaction of HIF-1 |
| inducible factor 1, | | | with transcriptional co- |
| alpha subunit | | | activators |
| inhibitor | | | |
| SERPINF1-Serpin | 2.3 | 9.2E−03 | Inhibitor of neovascularization |
| peptidase inhibitor, | | | and tumorigenesis and |
| clade F | | | activator of neurotrophic |
| | | | functions |
| THBS1- | 10.4 | 3.2E−03 | Inhibitor of neovascularization |
| Thrombospondin 1 | | | and tumorigenesis |

### Example 6: Modified Gene Expression Profile of si-hVDAC1-TTs

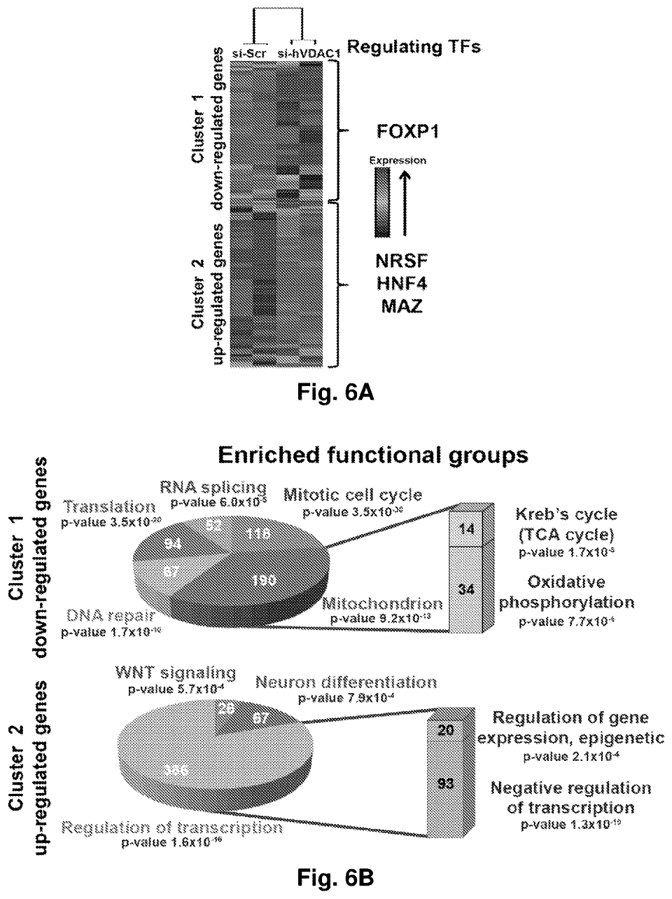

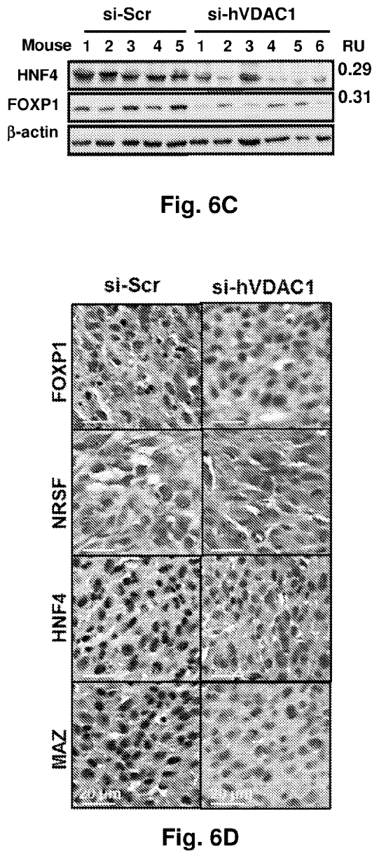

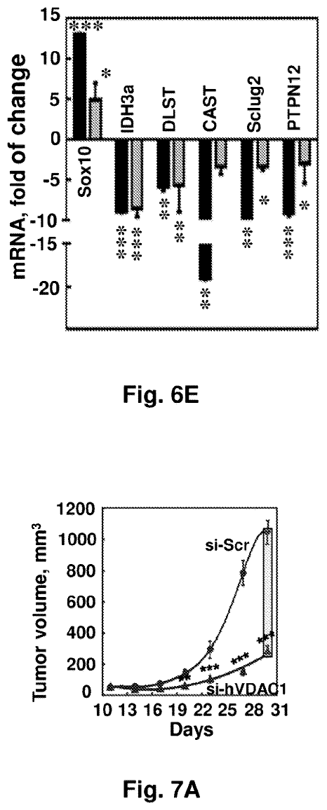

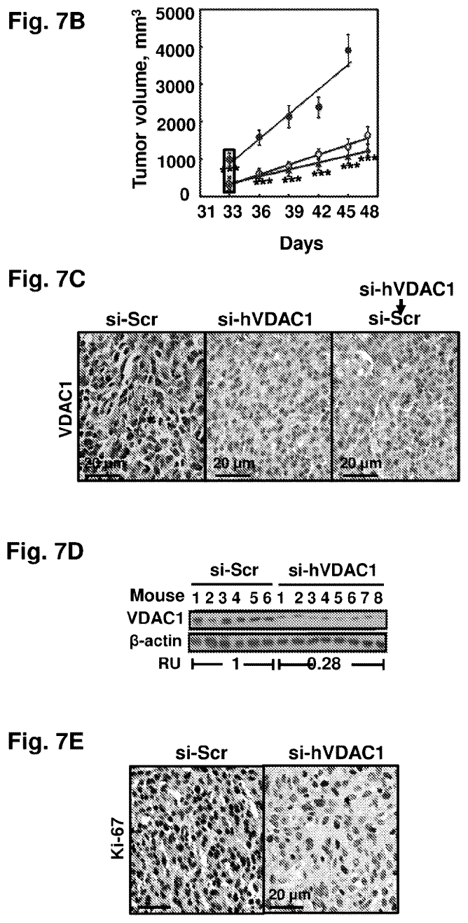

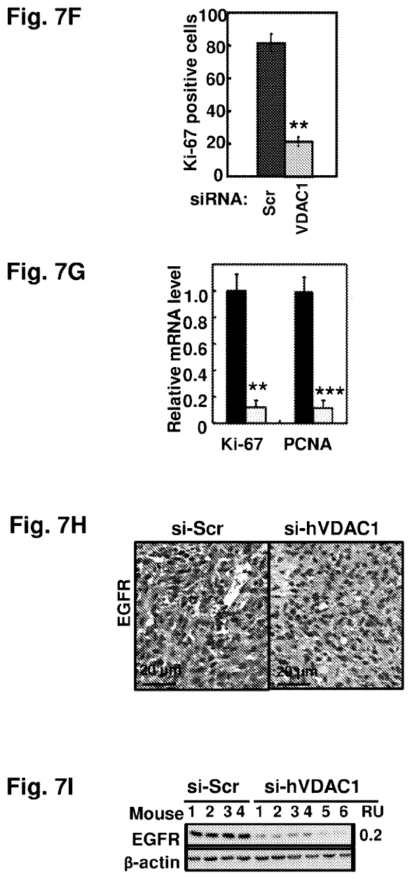

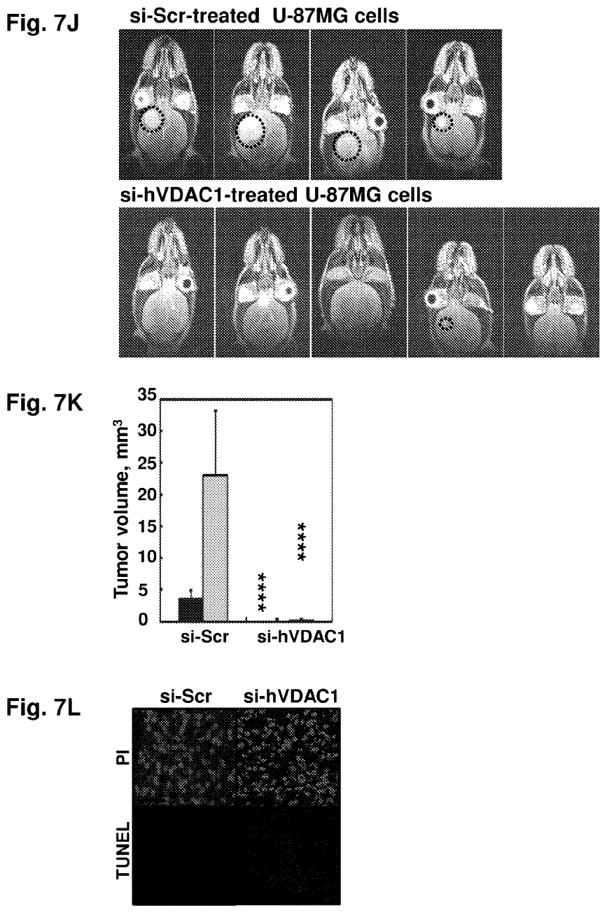

Affymetrix DNA microarray analysis was used to identify patterns of gene expression in si-hVDAC1- versus si-Scr-TTs (FIG. 6). Such analysis revealed 4,493 genes the expression of which was significantly changed (≥2-fold change, false discovery rate <0.05), which were clustered to 1,998 down-regulated (cluster 1) and 2,495 up-regulated genes (cluster 2) in the si-hVDAC1-TTs (FIG. 6A). Functional analysis based on the Gone Ontology (GO) system revealed alterations in key functions and pathways related to cancer tumorigenicity (FIG. 6B). The down-regulated group (FIG. 6B) was highly enriched for genes related to cell cycle, such as the cell cycle mitotic phase and DNA repair, and to the mitochondrion, such as Kreb's cycle and oxidative phosphorylation (OXPHOS), in agreement with IHC and WB results (data not shown). The up-regulated group (FIG. 6B) was enriched for genes related to the regulation of transcription and the WNT pathway. Remarkably, 67 genes were associated with neuronal differentiation.

Promoter analysis of the differentially expressed genes in si-hVDAC1-TTs revealed enrichment of several TF-binding sites (BSs). The down-regulated group showed high enrichment in genes encoding proteins with BSs for FOXP1 (Forkhead box protein P1). FOXP1 may act either as a transcriptional activator or repressor and as an oncogene or tumor suppressor (Katoh M et al., 2013. Cancer letters 328, 198-206). The analysis presented herein suggests that FOXP1 acts as a transcriptional activator and as an oncogene, as its level was down-regulated (FIG. 6C,D). In the up-regulated group, TF-BSs for three main TFs were highly enriched (FIG. 6A,B). NRSF (neuron-restrictive silencer factor), a transcriptional repressor of neuronal genes in non-neuronal cells (Lunyak V V and Rosenfeld M G., 2005. Cell 121, 499-50), was down-regulated in si-hVDAC1-TTs (FIG. 6D), allowing the cells to differentiate to neurons. The same was true for HNF4 (hepatocyte nuclear factor 4), a central regulator of hepatocyte differentiation and function (Watt A J et al., 2003. Hepatology 37, 1249-1253), and MAZ (myc-associated zinc-finger), which regulates c-Myc and HRAS (Cogoi et al., 2010). Together, HNF4 and MAZ co-regulate neural stem-cell differentiation (Wang et al., 2013). Down-regulation of HNF4 and MAZ in si-hVDAC1-TTs (FIG. 6C, D) is in agreement with differentiation of GBM cells in these tumors (FIG. 1).

Table 5 presents selected genes from the down-regulated (cluster 1) and up-regulated (cluster 2) groups (see also FIG. 5), representing major functional groups, i.e. associated with cell cycle, DNA repair, metabolism (TCA cycle, oxidative phosphorylation), regulation of gene expression, Wnt signaling or neuronal differentiation. For each gene, the gene symbol and name, linear fold of change in expression and p-value are indicated. Negative numbers represent down-regulation.

TABLE 5
List of selected genes differentially expressed between si-Scr- and si-
hVDAC1-treated tumors, as identified by DNA microarray analysis

| | si-hVDAC1- vs. | | |
|---|---|---|---|
| | si-Scr-treated | | |
| | tumors | | |
| | Fold- | | |
| Gene | Change | p-value | Function |

Cluster 1, down-regulated genes
Cellular metabolism and survival regulation

| | | | |
|---|---|---|---|
| EGFR-Epidermal growth | −2.78 | 4.9E−03 | EGF receptor, activates |
| factor receptor | | | the oncogenic Ras |
| | | | signaling cascade |
| MDM2-p53 binding | −7.97 | 2.2E−03 | ligase, regulates p53 |
| protein homolog | | | level via degradation |
| HIF1A-Hypoxia inducible | −13.34 | 2.2E−03 | Transcription factor, |
| factor 1, alpha subunit | | | master regulator of |
| | | | response to hypoxia |

Cell cycle

| | | | |
|---|---|---|---|
| CCNB1-cyclin B1 | −4.61 | 1.9E−03 | Essential for the control |
| | | | of the cell cycle at |
| | | | G2/M (mitosis) |
| CCNG1-cyclin G1 | −19.46 | 2.8E−05 | Associated with G2/M |
| | | | phase arrest in response |
| | | | to DNA damage in the |
| | | | TP53 pathway |

DNA repair

| | | | |
|---|---|---|---|
| ATM-serine/threonine | −3.10 | 3.8E−03 | Activates checkpoint |
| kinase | | | signaling upon double |
| | | | strand breaks |
| BRCA2-Breast cancer 2 | −4.20 | 8.9E−04 | Involved in double- |
| | | | strand break repair |
| | | | and/or homologous |
| | | | recombination |
| TP53BP1-Tumor protein | −3.10 | 1.3E−03 | Enhances TP53- |
| p53 binding protein 1 | | | mediated transcriptional |
| | | | activation. |
| TP53BP2-Tumor p53 | −2.35 | 1.6E−03 | Regulates TP53 by |
| binding protein 2 | | | enhancing its DNA |
| | | | binding |

TCA cycle

| | | | |
|---|---|---|---|
| IDH3A-Isocitrate | −9.00 | 6.1E−04 | TCA cycle enzyme |
| dehydrogenase 3 alpha | | | involved in energy |
| subunit | | | production |
| SDHB-Succinate | −4.86 | 4.5E−03 | Involved in energy |
| dehydrogenase complex, | | | production |
| subunit B, | | | |

Oxidative phosphorylation

| | | | |
|---|---|---|---|
| NDUFB6-NADH | −4.69 | 3.0E−04 | Electron transport |
| dehydrogenase | | | component, energy |
| (ubiquinone) 1 beta | | | production |
| sub-complex, 6, | | | |
| ATP5B-ATP synthase, | −2.77 | 1.0E−03 | Uses mitochondrial ΔΨ |
| beta subunit | | | for ATP synthesis |
| COX6C-Cytochrome c | −11.29 | 2.5E−03 | Electron transport |
| oxidase, subunit VIc | | | component, energy |
| | | | production |

Cluster 2, up-regulated genes
Negative regulation of gene expression

| | | | |
|---|---|---|---|
| ID4-Inhibitor of DNA | 6.10 | 4.4E−03 | Does not bind DNA |
| binding 4 | | | directly, rather inhibits |
| | | | activity of other |
| | | | transcription factors |
| FOXP4-Forkhead box P4 | 5.20 | 9.2E−03 | Transcriptional |
| | | | repressor that represses |
| | | | lung-specific |
| | | | expression |

Wnt signaling

| | | | |
|---|---|---|---|
| FZD1-Frizzled homolog 1 | 4.47 | 8.2E−03 | Receptor for Wnt |
| | | | proteins |
| WNT1-Wingless-type | 6.19 | 4.8E−03 | Ligand for members of |
| MMTV integration site | | | the frizzled family |
| family, member 1 | | | receptors |

Neuron differentiation

| | | | |
|---|---|---|---|
| UNC5B-HOMOLOG B, | 2.88 | 1.4E−03 | A netrin-1 receptor, |
| NETRIN RECEPTOR | | | thought to mediate the |
| | | | netrin-1 |
| | | | chemorepulsive effect |
| CDK5R1-cyclin- | 6.11 | 3.5E−04 | Involved in proper |
| dependent kinase 5, | | | development of the |
| regulatory subunit 1 (p35) | | | central nervous system |
| MANF-Mesencephalic | 4.48 | 9.2E−04 | Involved in ER stress- |
| astrocyte-derived | | | induced death and cell |
| neurotrophic factor | | | proliferation |

### Example 7: VDAC1 Depletion Revers Reprogrammed Cancer Cell Metabolism

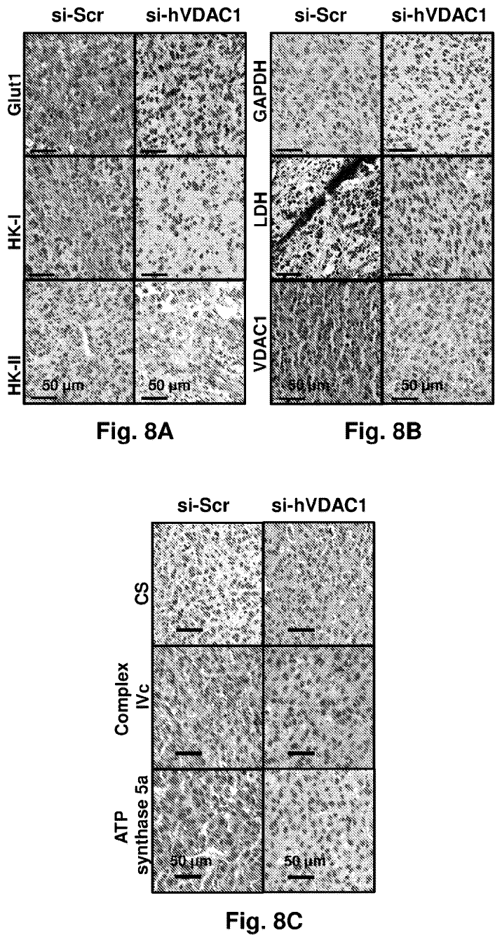

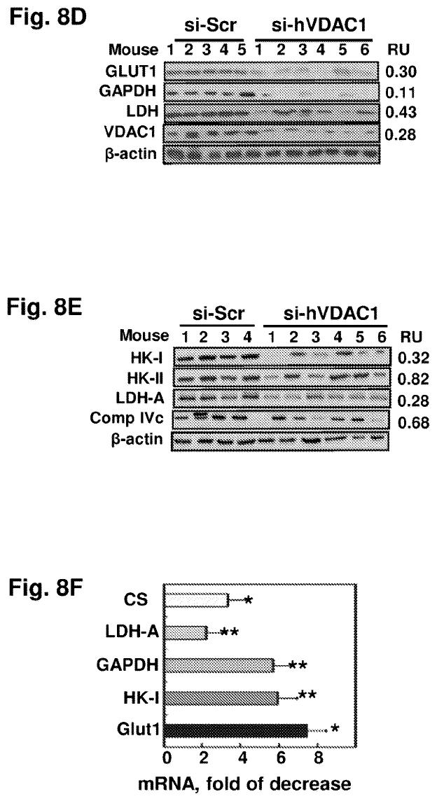

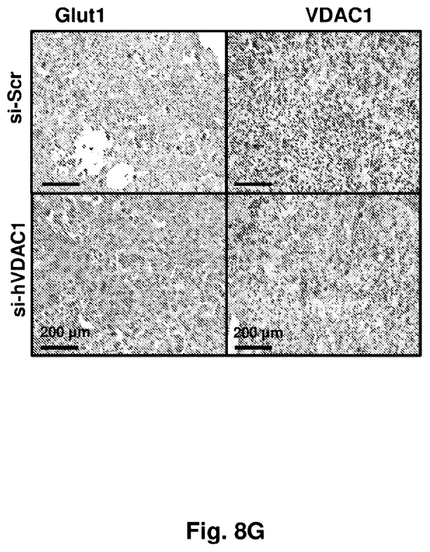

The metabolic alterations that occur during malignant transformation involve a spectrum of functional aberrations and mutations which contribute to elevated glycolysis and increased expression levels of glucose transporters (Glut) and glycolytic enzymes (Koppenol W H et al., 2011. Nature reviews Cancer 11, 325-337) (FIG. 8). si-hVDAC1-TTs showed dramatic decreases of Glut-1, hexokinase (HK-1), glyceraldehyde dehydrogenase (GAPDH) and lactate dehydrogenase (LDH) levels, as compared to si-Scr-TTs (FIG. 8A,B,D,E). Expression levels of the Kreb's cycle enzyme, citrate synthase (CS), the mitochondrial electron transport complex IVc. and ATP synthase 5a were also highly reduced in si-hVDAC1-TTs (FIG. 8C,E), consistent with alterations in oxidative phosphorylation (OXPHOS) Similar results were obtained by qRT-PCR (FIG. 8F). The decreased expression of Kreb's cycle and OXPHOS enzymes is in agreement with the concept that cancer cells use a combination of glycolysis and mitochondria to produce energy, reflecting prevalent normoxic or hypoxic condition. Glioma cells also show similar metabolic flexibility. Similar results were obtained with U-118 xenografts (data not shown). In the orthotopic model, tumors derived from si-Scr-treated U-87MG cells expressed high levels of Glut1. VDAC1 and LDH but not CS or ATP synthase. Neither tumors nor modified expression of these proteins were seen in brain inoculated with si-hVDAC1-treated cells (FIG. 8G). Together, these findings thus point to a reversal of the metabolic reprogramming of cancer cells upon V DAC1.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1-6. (canceled)

7. A method for inducing the transition of a cancer stem cell and/or quasi differentiated cancer cell into a differentiated cell, the method comprising administrating to, or expressing in the cancer stem cell or quasi differentiated cancer cell an effective amount of a voltage-dependent anion channel 1 (VDAC1)-silencing oligonucleotide, comprising the nucleic acid sequence set forth in SEQ ID NO:10 and a complementary sequence thereto.

8. The method of claim 7, wherein the VDAC1-silencing oligonucleotide is a dsRNA comprising the nucleic acid sequence set forth in SEQ ID NO:10 and the complementary sequence comprising the nucleic acid sequence set forth in SEQ ID NO:13.

9. The method of claim 7, wherein the cancer stem cell and/or quasi differentiated cancer cell form part of a tumor of a subject.

10. The method of claim 7, wherein the cancer stem cell and/or quasi differentiated cancer cell is within a tumor, a tissue, or a cell culture.

11. The method of claim 8, wherein the cancer stem cell and/or quasi differentiated cancer cell form part of a tumor of a subject.

12. The method of claim 8, wherein the cancer stem cell and/or quasi differentiated cancer cell is within a tumor, a tissue, or a cell culture.
